SEQUENCE LISTING (37 sequences)

SEQ ID NO: 1 - DNA, 22 bp, Artificial Sequence
  Dyf7-CT-sense oligonucleotide
ccgtcgtaagcagaattgaatc

SEQ ID NO: 2 - DNA, 22 bp, Artificial Sequence
  Dyf7-CT-antisense oligonucleotide
catcatgtcaaaggagtccttc

SEQ ID NO: 3 - DNA, 22 bp, Artificial Sequence
  Dyf7-CeB-sense oligonucleotide
gtcgaaacagtaaatgaaagac

SEQ ID NO: 4 - DNA, 18 bp, Artificial Sequence
  Dyf7-CeB-antisense oligonucleotide
ctcatttctggtcaaacg

SEQ ID NO: 5 - DNA, 29 bp, Artificial Sequence
  Hc-dyf7-1-for oligonucleotide
ttggagtcaaaatcgactgcgacggcatt

SEQ ID NO: 6 - DNA, 29 bp, Artificial Sequence
  Hc-dyf7-1-rev oligonucleotide
tcaccaaagccgtcggtatgtgcttgtcg

SEQ ID NO: 7 - DNA, 29 bp, Artificial Sequence
  Hc-dyf7-2-for oligonucleotide
ttggagtcaaaatcgactgcgacggcatt

SEQ ID NO: 8 - DNA, 29 bp, Artificial Sequence
  Hc-dyf7-2-rev oligonucleotide
tgtgaggcttcccgacgagcatgacacct

SEQ ID NO: 9 - DNA, 23 bp, Artificial Sequence
  Hc-dyf7-3-for oligonucleotide
tgcattactgtagcccaggctct

SEQ ID NO: 10 - DNA, 22 bp, Artificial Sequence
  Hc-dyf7-3-rev oligonucleotide
accggttgcgattttgggtcgt

SEQ ID NO: 11 - DNA, 23 bp, Artificial Sequence
  Hc-dyf7-4-for oligonucleotide
tctttccagtggacgaggtgtca

SEQ ID NO: 12 - DNA, 23 bp, Artificial Sequence
  Hc-dyf7-4-rev oligonucleotide
agaggtcgtccatcagtgcttct

SEQ ID NO: 13 - DNA, 24 bp, Artificial Sequence
  Hc-dyf7-5-for oligonucleotide
gcgctcgaagacaatgtggatact

SEQ ID NO: 14 - DNA, 23 bp, Artificial Sequence
  Hc-dyf7-5-rev oligonucleotide
acacttgtaccgtgcactgagcg

SEQ ID NO: 15 - DNA, 20 bp, Artificial Sequence
  Hc-dyf7-6-for oligonucleotide
tgctatcacccgatgcccga

SEQ ID NO: 16 - DNA, 25 bp, Artificial Sequence
  Hc-dyf7-6-rev oligonucleotide
ccttcttctgtttccatgtcgtcca

SEQ ID NO: 17 - DNA, 23 bp, Artificial Sequence
  Hc-dyf7-7-for oligonucleotide
agcagtgaatccgccttttagca

SEQ ID NO: 18 - DNA, 23 bp, Artificial Sequence
  Hc-dyf7-7-rev oligonucleotide
agcattagcggagctggaatccg

SEQ ID NO: 19 - DNA, 27 bp, Artificial Sequence
  Hc-dyf7-8-for oligonucleotide
tgtgttgggacccgattcttagctagt

SEQ ID NO: 20 - DNA, 27 bp, Artificial Sequence
  Hc-dyf7-8-rev oligonucleotide
ccgaaggtgacagacaatgtcaaatgt

SEQ ID NO: 21 - DNA, 20 bp, Artificial Sequence
  Dyf7-ex4-5-sense oligonucleotide
agccgaaaccgaaagtagag

SEQ ID NO: 22 - DNA, 21 bp, Artificial Sequence
  Dyf7-ex4-5-antisense oligonucleotide
cgctgaacatgcactcttttg

SEQ ID NO: 23 - DNA, 24 bp, Artificial Sequence
  Dyf7-sense oligonucleotide
ggtttggcaatcaaagcaccgtct

SEQ ID NO: 24 - DNA, 22 bp, Artificial Sequence
  Dyf7-antisense oligonucleotide
aaaggcggattcactgctctct

SEQ ID NO: 25 - DNA, 1173 bp, Haemonchus contortus
atgatactaatagtactatttggtcttcttcgacaagcacataccgacggctttgtggacgaggtgtcatgctcgtcgggaagcctcacagtcatgctcaacaagtcggatccagacattgcccggtggatgaacgacccaaaatcgcaaccggtggtgtatgtctacggtcacaagacacttgtgccttgtggaacatcattaaaaaacgacaaaggacaacaaaattacaatctgacgataccttacgggaaacattgtgatgtccatcttgctgacctggaaccgaactaccgaacagcggagactacgatagcgctcgaagacaatgtggatactagtatatccaaagtaattcggatcaatcacgtgttctgtctctacacacgaagtgtccagactatcagatacagtgatgtatcgacggcccatgaagtgttggcatcgactggaggcaagccgaaaccgaaagtagagatgatgtttagaagcactgatggacgacctctgagaacagcgaaattcggcgatactgtagaattttacgttgctctgacacctgataaggcgtaccacggtatctcaccaaaagagtgcatgttcagcgaccgtgaggatatgctatcacccgatgcccgacatctcacatttgttcaaagcagttgtccagtacatgaaatgtccgagattatcgatccgttggcaaatgttaacgaggaagtctatttttcaaagttcaaaacgttccggtttggcaatcaaagcaccgtcttcgctcagtgcacggtacaagtgtgcctcgttggatcggaatgcgttcagaattgtttcaaacgtatttcaaattcgaatttgacggcagaacgacttcgatttcgtcataagagagcagtgaatccgccttttagcaaagatgacgtatacgatcaagtctcagtgagagagtcactaacaattctggacgacatggaaacagaagaagtgtcatccagatcaacggagcaatgtttggtgtcgacttcacggattccagctccgctaatgcttttcatagtcatgctgttgattttctgcattgcatcggctagtgtagcagcctaccttgggtataggcttttccggaggagaaagcaaacatcactggacgtttattccgtgtactcatcatcgatg

SEQ ID NO: 26 - DNA, 1173 bp, Haemonchus contortus
atgatactaatagtactatttggtcttcttcgacaagcacataccgacggctttgtggacgaggtgtcatgctcgtcgggaagcctcacagtcatgctcaacaagtcggatccagacattgcccggtggatgaacgacccgaaatcgcaacctgtggtgtatgtctacggtcacaagacacttgtgccttgcggaacatcattaaaaaacgacaaagggcaacaaaattacaacctgacgataccatacgggaaacattgtgatgttcatcttgctgacctggaaccaaactaccgaacagccgagaccacaatagcgctcgaagacaatgtggatactagtatatccaaagtaattaggatcaatcacgtgttctgtctctacacacgaagtgttcagacaatcagatacagtgatgtatcgacggcccatgaagttttggcatcaactggaggcaagccaaaaccgaaagtagagatgatgttcagaagcacagatggacgacctctgagaacagctaaattcggcgatactgtcgaattttatgttgctctgacacctgataaggcgtaccacggtatctcgccaaaagaatgcatgttcagcgaccgtgaggatatgctatcacccgatgcccgacatctcacgtttgttcaaagcagttgtccagtgcatgaaatgtccgagattatcgatccattggcaaatgtcaacgaggaagtctatttttcaaagttcaaaacattccggtttggcaatcaaagcaccgtcttcgctcagtgcacggtacaggtgtgcctcgttggatcggaatgtgttcagaattgtttcaaacgtatttcaaattcgaatttgacggcagaacgacttcgatttcgtcataagagagcagtgaatccgccttttagcaaagatgacgtatacgatcaagtctcagtgagagagtcactaacaattctggacgacatggaaacagaagaagtgtcatccagatcaacggagcaatgtttggtgtcgacttcacggattccagctccgctaatgcttttcatagtcatgctgttgattttctgcattgcatcggctagtgtagcagcctaccttgggtataggcttttccggaggagaaagcaaacatcactggacgtttattccgtgtactcatcatcgatg

SEQ ID NO: 27 - DNA, 4804 bp, Haemonchus contortus
atttcgctttttttgtaagtgaggaaaaggttcttattatgcagtcctgatcggatcattaaatccatgagcgtgtctatttacaaggtccgattcgatgaatccgtactttttcctgccacttcctgttatttaatccattatttcagaatttatgatactaatagtactatttggtcttcttcgacaagcacataccgacggctttggtgaggagaagcaatatttactcttttttcttaatcagaactggtaaactttcggtatttgaattcaatttgcttacgaagtacctagaaagttgttcttcaggactattaggaagatttacttcagtttgtttttaagtaattttgcaaaaagaatcaatacgcccaccacaaatcaaatcggatgtccctttcataagagaacggggtacttacagatctatgaaaaagctgagcggccttcggaaggagtgtttttcagtgatggaagtcatcctttatggttggcataagagtaatcgcatgcataataaatcagcctgtgatctcctataaacaaaagggataaagtaaagtgaatcggtgtcagggacctagaaatcagaactgctcaatagcagggggacttcgtacattcagaatgattggaattgcatctagacattttacctacttttaggttattcttagtaagctgctgtgaatatgaattcccatagaagggtctgcaacccctccccttcaagttgaatctcttgcccttgtttttggtgacctttgcagtcacaactttgccgaatcggggatatcatgacccagctcaatgacatgcagcagccagtgctaagtggaatggcgtctccattgttaagttccactgggtctcactagcaatcctaagtaaaattctataaacctttgcaaattctattgcatgggttgggtactgtgtattgtattacttcttcaattgctagcctgcaaacacgttttcgaacaaaatgaaacgactgacctggtctccaacagcaaattctaaaaaattcccgtttttattgcagtgctctgcgctttacctaattggtgctcgtaggcaggccaccattcacaaaccttccagagtccaacaagcgtagcactattttagctcatcagacgaaggctaggcttacctgatattttttgaacatggtgttaatgcactccactagtaaagcaataacgacttaaaagtgtttgctcttgaggaatcgaccagaatgttaacgatttcacctactgctactacttcggggttaacgattcacttctaactgatttctgcattactgtagcccaggctctgagcagtgggaacgatgagtgtactacaaatataagtaatattataaatcgcgttgtaaatattatcctaagcgcgttgctcattggttgcatgtcaaactacgcgaccgtaacatgttttattgccaacaatccaccaataagtgcactgaacccgttagctccgcctccgtacgtaattgcactgcgctaagggtatcgtagcgttaaatagagtgcatcccaattgcgtaatcttgggaaatggcatgtaaggacgtggcacccatcaacaaagatcttactgtttagaactagagtaaagattcaagagtaatcatacttttcccttgagttctttctttccagtggacgaggtgtcatgctcgtcgggaagcctcacagtcatgctcaacaagtcggatccagacattgcccggtggatgaacgacccaaaatcgcaaccggtggtaagcgaaccttttaagttgatgagtaacgcttcatagacatgcggaaattcgagctgtactagttacctggtgtcagagtaattcaagtagtattttaggtgtatgtctacggtcacaagacacttgtgccttgtggaacatcattaaaaaacgacaaaggacaacaaaattacaatctgacgataccttacgggaaacattgtgatgtccatcttgctgacctggtttgttagcatctgttgaccatcaatatcaagcaatattgatgggtattgattgattccccattagggaggaattgatttgctcctggattgaccagctactttaggaaccgaactaccgaacagcggagactacgatagcgctcgaagacaatgtggatactagtatatccaaagtaattcggatcaatcacgtgttctgtctctacacacgaagtgtccagactatcagatacagtgatgtatcgacgttagtgctacctaggtccacgtttgatcattcaggcttcggaatgaaaatttcctgttttccagggcccatgaagtgttggcatcgactggaggcaagccgaaaccgaaagtagagatgatgtttagaagcactgatggacgacctctgagaacagcgaaattcggcgatactgtagaattttacgttgctctgacacctgatagtaagtagcctttgttttagcgaaatgaatgtcatcctgctatacggtagcacattacggtaatttgttttcagaggcgtaccacggtatctcaccaaaagagtgcatgttcagcgaccgtgaggatatgctatcacccgatgcccgacatctcacatttgttcaaagcaggtgagtatagagaccagaaggaatgacgttccaaaggccgtcctcttcaatttttcccgtttcagttgtccagtacatgaaatgtccgagattatcgatccgttggcaaatgttaacgaggaagtctatttttcaaagttcaaaacgttccggtttggcaatcaaagcaccgtcttcgctcagtgcacggtacaagtgtgcctcgttggatcggaatgcgttcaggtaagcgtggcaaagaccaaacctttcaagaaccctccagctgatgtataataattcaaatttacttagaacatcccgaaagagggttttttcacttatttcacttaaaattcaagccatttttaatccaaatagagaaaccaaaccaaataaaaacataccgggaagttcggctgatctcgttctgtggtaaggtaccttgttagaaaggaaagaggggtaccaccaagccatttttcatgtatggtgttattgccaatgaaacagggtagatggagctgtatataggtaaaaacttctgaattggtgattttcaccttcaaaatgctcgaaaacaaagttcgttcccaaatggggctcgtgaaaccattccccatgcccttccgttcccaaaaaccctgttcttgtcagccttgcttcttccccacgtattctaaccaacgctttgaattttagaaatgttctatatttggctataaaactcccgtttcaagcagagaatcataaaattccattaaatttcccatttcagaattgtttcaaacgtatttcaaattcgaatttgacggcagaacgacttcgatttcgtcataagagagcagtgaatccgccttttagcaaagatgacgtatacgatcaagtctcagtgagagagtcactaacaattctggacgacatggaaacagaagaaggtgcttatttgtattctcaggttgaagagctaccattctgtccgatgcctaatcaataaggaagcctacccgcgactggaactccccaattgatcgcttggtaccaatgtagaaattcacggggtatcggcttatagatcaagctaaacttttcggctgcacccattacagtccaaagaactttttcttagctctagagtgggaaggggggaactttcacctactacactcggatctggcataagtttttgggataatcgaatatcagtatgtatgagttgagtatgagtcgtcaagcgatacgcgggacgtgcggcatctacatcaaatactcgtactcgagtgtgttgggacccgattcttagctagttgtcccaaaccgcggcatgtcttggttcgctacccaaagctccgctaatgttattctagcgacaatgcctattggaacacccaagataaaacttttccttttaaaaatgcaggcgaaaatactttaacatcatcgacttttcagtgtcatccagatcaacggagcaatgtttggtgtcgacttcacggattccagctccgctaatgcttttcatagtcatgctgttgattttctgcattgcatcggctagtgtagcagcctaccttgggtataggcttttccggaggagaaaggttcgtccgactttgaaaattttgtcatattggtcccagagcacgaaaaacagacttggatggtaacctagaagttcagagtgtcccccggccagcgcagggatatttaactgcaagggttttacaatatttaaatcttttcggggaacagggtagttgaatcggatttgtgtaatcgattcgccgcacgcggctgccgcgtactagttgtcttgctattgcagttctaacgttcttcgtagagctcgaccgatatgaattactatttttgccataattttgttgttacaagatttgttattgaattccttactcgctacgctgcttccgcttcattctgcgaaattccttcgtgcgcgacgtcgctctcgccgcgaaacgaccactttgatttaactgtaccacaccctcctttccgtatggataggtgctggcgctggctagggagttattctaagacttgaagagcttctctgataaaatttctgactatttcaccaaggaacttgactagttagttttgtaagttcaaatatttttgttaagcaaacatcactggacgtttattccgtgtactcatcatcgatg

SEQ ID NO: 28 - DNA, 5126 bp, Haemonchus contortus
atttcgctttttttgtaagtgaggaaaaggttcttattatgcagtcctgatcggatcattaaatccatgagcgtgtctatttacaaggtccgattcgatgaatccgtactttttcctgccacttcctgttatttaatccattatttcagaatttatgatactaatagtactatttggtcttcttcgacaagcacataccgacggctttggtgaggagaagcaatatttactcttttttcttaatcagaactggtaaactttcggtatttgaattcaatttgcttacgaagtacctagaaagttgttcttcaggactattaggaagatttacttcagtttgtttttaagtaattttgcaaaaagaatcaatacgcccaccacaaatcaaatcggatgtccctttcataagagaacggggtacttacagatctatgaaaaagctgagcggccttcggaaggagtgtttttcagtgatggaagtcatcctttatggttggcataagagtaatcgcatgcataataaatcagcctgtgatctcctataaacaaaagggataaagtaaagtgaatcggtgtcagggacctagaaatcagaactgctcaatagcagggggacttcgtacattcagaatgattggaattgcatctagacattttacctacttttaggttattcttagtaagctgctgtgaatatgaattcccatagaagggtctgcaacccctccccttcaagttgaatctcttgcccttgtttttggtgacctttgcagtcacaactttgccgaatcggggatatcatgacccagctcaatgacatgcagcagccagtgctaagtggaatggcgtctccattgttaagttccactgggtctcactagcaatcctaagtaaaattctataaacctttgcaaattctattgcatgggttgggtactgtgtattgtattacttcttcaattgctagcctgcaaacacgttttcgaacaaaatgaaacgactgacctggtctccaacagcaaattctaaaaaattcccgtttttattgcagtgctctgcgctttacctaattggtgctcgtaggcaggccaccattcacaaaccttccagagtccaacaagcgtagcactattttagctcatcagacgaaggctaggcttacctgatattttttgaacatggtgttaatgcactccactagtaaagcaataacgacttaaaagtgtttgctcttgaggaatcgaccagaatgttaacgatttcacctactgctactacttcggggttaacgattcacttctaactgatttctgcattactgtagcccaggctctgagcagtgggaacgatgagtgtactacaaatataagtaatattataaatcgcgttgtaaatattatcctaagcgcgttgctcattggttgcatgtcaaactacgcgaccggtaacatgttttattgccaacaatccaccaataagtgcactgaacccgttagctccgcctccgtacgtaattgcactgcgctaagggtatcgtagcgttaaataragtgcatcccaattgcgtaatcttgggaaatggcatgtaaggacgtggcacccatcaacaaagatcttactgtttagaactagagtaaagattcaagagtaatcatacttttcccttgagttctttctttccagtggacgaggtgtcatgctcgtcgggaagcctcacagtcatgctcaacaagtcggatccagacattgcccggtggatgaacgacccgaaatcgcaacctgtggtcagtcaaccttcctatgttaatgagtactttactcacaggcatgaggaaatgcaaatcgtactagatacctggtgtaagagtaatttagatagtattttaggtgtatgtctacggtcacaagacacttgtgccttgcggaacatcattaaaaaacgacaaagggcaacaaaattacaacctgacgataccatacgggaaacattgtgatgttcatcttgctgacctggtttgttagcatctgttggccatcaataccaagcaatattgattagtattgattgattccccattagggatgagtttactcctggattgagcagctactttaggaaccaaactaccgaacagccgagaccacaatagcgctcgaagacaatgtggatactagtatatccaaagtaattaggatcaatcacgtgttctgtctctacacacgaagtgttcagacaatcagatacagtgatgtatcgacgttagtactacctaattccatgtttgatcattcaggtttcggaatgaaaattcactgttttccagggcccatgaagttttggcatcaactggaggcaagccaaaaccgaaagtagagatgatgttcagaagcacagatggacgacctctgagaacagctaaattcggcgatactgtcgaattttatgttgctctgacacctgatagtaagtagactttgtttttcgtgaaatgaatgttgttctatcatacggtagcacattacggtaatttgtttacagaggcgtaccacggtatctcgccaaaagaatgcatgttcagcgaccgtgaggatatgctatcacccgatgcccgacatctcacgtttgttcaaagcaggtgagtatagagaccaaaagaaaggccgttctcggagtccattctcttttattttcccgtttcagttgtccagtgcatgaaatgtccgagattatcgatccattggcaaatgtcaacgaggaagtctatttttcaaagttcaaaacattccggtttggcaatcaaagcaccgtcttcgctcagtgcacggtacaggtgtgcctcgttggatcggaatgtgttcaggtgagttcagcgtgggccaaacctctaaagggccccacaactgatattattactgaaattgaattagaacatcgcgaaacaggagttcatttttcacttcttccactggaaattgaagccccttttcaaattgagtaccaaaatcaaacaaatgcgaccaaataaaaaaatacggggtaactagtttgatctcgatttgtggtaaagaaaactggttagaaaggaaaagggctaccactaggtcatcttccatatacgatgatattaccaatgaaaaagggtggatgaagctccatacaggtaaaaactcgtaaactggtgattttcaccttcatcatactcaaaaacaatgttcggcttctaactggggtttatgaaacaattcctcatgtcccttcattcccggggaccctgtttggttcagcattgcttccttccatgtattctaaccaacgctttgaattttagaaatgttctatatttggccataaaactcccgtttcaagcagggaatcataaaatcccattaaatttcccatttcagaattgtttcaaacgtatttcaaattcgaatttgacggcagaacgacttcgatttcgtcataagagagcagtgaatccgccttttagcaaagatgacgtatacgatcaagtctcagtgagagagtcactaacaattctggacgacatggaaacagaagaaggtgcwtatttgtattcctaggttggatagctaccattctctccgatgcctaatcaataaagaagcctaaccgcgacaggaactccccaattgatcgcttagtaccaaaagtaaaacttaagtagatgtcggctcattgatcaagctcaatctttcgactgcacccatcatcctatcgctgatagcctttggatagcacctagcgagaaaaagaatggaactgttactttactgcaattgatgtcggtataggtcgccgcacgccacgcactccgcttaacaacctataagcggaactgattgccggttatcctttgtgatattagtagacgattctctgaaacctatgcaggcccctagtatgctagggtctttcataacttttcggcgctaatcatgggtgaacattccttgaatgaagaggcaacagaatgggagtcgtctacaaatgtcatggaggataattggtaatcagtaccggttactggtcttcaagcgaaatgcgtggcgtacctatgcctacatcggagctcgcccttggttatgtagggaacccaaatgcagccagttgtcccagacmgcgtcatgtcttaccagggaagattttcaacttcccatggaacctcgaaaagagacctacgtatatccatggagtttggtatgctatttacaagtttagaagttagaattttcttcagtgatcagttaattgcccaaagttgctctattatagtttgcgcggctcgccaggtatgcggagccaccaaagccactctttgtctcttaagaatgcgcacgaaatactctatcactatcacttttcagtgtcatccagatcaacggagcaatgtttggtgtcgacttcacggattccagctccgctaatgcttttcatagtcatgctgttgattttctgcattgcatcggctagtgtagcagcctaccttgggtataggcttttccggaggagaaaggttcgtccgactttgaaaattttgtcatattggtcccagagcacgaaaaacagacttggatggtaacctagaagttcagagtgtcccccggccagcgcagggatatttaactgcaagggttttacaatatttaaatcttttcggggaacagggtagttgaatcggatttgtgtaatcgattcgccgcacgcggctgccgcgtactagttgtcttgctattgcagttctaacgttcttcgtagagctcgaccgatatgaattactatttttgccataattttgttgttacaagatttgttattgaattccttactcgctacgctgcttccgcttcattctgcgaaattccttcgtgcgcgacgtcgctctcgccgcgaaacgaccactttgatttaactgtaccacaccctcctttccgtatggataggtgctggcgctggctagggagttattctaagacttgaagagcttctctgataaaatttctgactatttcaccaaggaacttgactagttagttttgtaagttcaaatatttttgttaagcaaacatcactggacgtttattccgtgtactcatcatcgatg

SEQ ID NO: 29 - DNA, 590 bp, Artificial Sequence
  Dyf7 fragment amplified from the Bristol N2 strain
tgcataaaaccctagaaatgaattaattaaacttattttagttgttgcttccccgatttcgcaaccaacaattgtcgatacaccaagtgagcaaagaagagacccatgtccaaagagctcaaatatgggattcatcccactcatcatcatgggctctcttgcgtctttgttactattctccgcaggcgctgcaatttactttgggtgtaaactgaaaagtatgaaaaaggtatgttgaaaaaaagtaactagctggctgagtatttgaaaccgttttttagttgtaatcgttagtgggaatttcgtataattacacaaatagtagtccaataaaaacggatgtcataatcagaaaaaattaattttattcaggttttatgttcaaactcccacaattttcaattaaaactatttaaatcgttgtctttgaaaataaatgactaacctatgaatagttttagacatcttgaacagttgaacacaatttcaaacagaagagtaggttgatttatttgataaaaactgtttaaagcattcacattataaccatttcaaagtgtcaattccaaaaatgctgttatgaacaactg

SEQ ID NO: 30 - DNA, 588 bp, Artificial Sequence
  Dyf7 fragment amplified from the IVR6 strain
tgcataaaaccctagaaatgaattaattaaacttattttagttgttgcttccccgatttcgcaaccaacaattgtcgatacaccaagtgagcaaagaagagacccatgtccaaagagctcaaatatgggattcatcccactcatcatcatgggctcttgcgtctttgttactattctccgcaggcgctgcaatttactttgggtgtaaactgaaaagtatgaaaaaggtatgttgaaaaaaagtaactagctggctgagtatttgaaaccgttttttagttgtaatcgttagtgggaatttcgtataattacacaaatagtagtccaataaaaacggatgtcataatcagaaaaaattaattttattcaggttttatgttcaaactcccacaattttcaattaaaactatttaaatcgttgtctttgaaaataaatgactaacctatgaatagttttagacatcttgaacagttgaacacaatttcaaacagaagagtaggttgatttatttgataaaaactgtttaaagcattcacattataaccatttcaaagtgtcaattccaaaaatgctgttatgaacaactg

SEQ ID NO: 31 - DNA, 590 bp, Artificial Sequence
  Dyf7 fragment amplified from the IVR10 strain
tttgcataaaaccctagaaatgaattaattaaacttattttagttgttgcttccccgatttcgcaaccaacaattgtcgatacaccaagtgagcaaagaagagacccatgtccaaagagctcaaatatgggattcatcccactcatcatcatgggctcttgcgtctttgttactattctccgcaggcgctgcaatttactttgggtgtaaactgaaaagtatgaaaaaggtatgttgaaaaaaagtaactagctggctgagtatttgaaaccgttttttagttgtaatcgttagtgggaatttcgtataattacacaaatagtagtccaataaaaacggatgtcataatcagaaaaaattaattttattcaggttttatgttcaaactcccacaattttcaattaaaactatttaaatcgttgtctttgaaaataaatgactaacctatgaatagttttagacatcttgaacagttgaacacaatttcaaacagaagagtaggttgatttatttgataaaaactgtttaaagcattcacattataaccatttcaaagtgtcaattccaaaaatgctgttatgaacaactg

SEQ ID NO: 32 - DNA, 590 bp, Artificial Sequence; identical to SEQ ID NO: 31
  Consensus sequence shown on Figure 1

SEQ ID NO: 33 - DNA, 1173 bp, Haemonchus contortus; identical to SEQ ID NO: 25

SEQ ID NO: 34 - DNA, 1173 bp, Haemonchus contortus; identical to SEQ ID NO: 26

SEQ ID NO: 35 - DNA, 4804 bp, Haemonchus contortus; identical to SEQ ID NO: 27

SEQ ID NO: 36 - DNA, 5126 bp, Haemonchus contortus; identical to SEQ ID NO: 28

SEQ ID NO: 37 - DNA, 1173 bp, Artificial Sequence
  Consensus sequence presented in Figure 4
atgatactaatagtactatttggtcttcttcgacaagcacataccgacggctttgtggacgaggtgtcatgctcgtcgggaagcctcacagtcatgctcaacaagtcggatccagacattgcccggtggatgaacgacccaaaatcgcaaccggtggtgtatgtctacggtcacaagacacttgtgccttgcggaacatcattaaaaaacgacaaaggacaacaaaattacaacctgacgataccatacgggaaacattgtgatgtccatcttgctgacctggaaccaaactaccgaacagccgagaccacaatagcgctcgaagacaatgtggatactagtatatccaaagtaattaggatcaatcacgtgttctgtctctacacacgaagtgtccagacaatcagatacagtgatgtatcgacggcccatgaagtgttggcatcaactggaggcaagccaaaaccgaaagtagagatgatgttcagaagcacagatggacgacctctgagaacagcgaaattcggcgatactgtagaattttacgttgctctgacacctgataaggcgtaccacggtatctcaccaaaagaatgcatgttcagcgaccgtgaggatatgctatcacccgatgcccgacatctcacatttgttcaaagcagttgtccagtacatgaaatgtccgagattatcgatccattggcaaatgtcaacgaggaagtctatttttcaaagttcaaaacattccggtttggcaatcaaagcaccgtcttcgctcagtgcacggtacaagtgtgcctcgttggatcggaatgcgttcagaattgtttcaaacgtatttcaaattcgaatttgacggcagaacgacttcgatttcgtcataagagagcagtgaatccgccttttagcaaagatgacgtatacgatcaagtctcagtgagagagtcactaacaattctggacgacatggaaacagaagaagtgtcatccagatcaacggagcaatgtttggtgtcgacttcacggattccagctccgctaatgcttttcatagtcatgctgttgattttctgcattgcatcggctagtgtagcagcctaccttgggtataggcttttccggaggagaaagcaaacatcactggacgtttattccgtgtactcatcatcgatg

(19) United States
(12) Patent Application Publication
(10) Pub. No.: US 2015/0017636 A1
(43) Pub. Date: Jan. 15, 2015

(54) Detection and Monitoring of Macrocyclic Lactone Resistance in Nematodes

(71) Applicant: THE ROYAL INSTITUTION FOR THE ADVANCEMENT OF LEARNING/MCGILL UNIVERSITY, Montreal (CA)

(72) Inventors: Roger Prichard, Sainte-Anne-de-Bellevue (CA); Ludmel Urdaneta-Marquez, Verdun (CA); Joseph Dent, Montreal (CA)

(21) Appl. No.: 14/328,412

(22) Filed: Jul. 10, 2014

Related U.S. Application Data
(60) Provisional application No. 61/845,246, filed on Jul. 11, 2013.

(51) Int. Cl. C12Q 1/68

(52) U.S. Cl. CPC C12Q 1/6888; C12Q 2600/156; C12Q 2600/142; C12Q 2600/124; C12Q 2600/106
USPC 435/6.11

(57) ABSTRACT

The present disclosure concerns the determination of the resistance or the susceptibility of a nematode to a macrocyclic lactone anti-helminthic based on the characterization of the Dyf-7 gene (or Dyf-7 gene ortholog) or its corresponding gene products. The present disclosure also provides tools and commercial packages for making such characterization.

### CROSS-REFERENCE TO RELATED APPLICATION AND DOCUMENTS

The application claims priority from U.S. provisional patent application 61/845,246 filed on Jul. 11, 2013 and is incorporated herewith in its entirety. This application also contains a sequence listing in electronic format filed separately and the content of such sequence listing is also incorporated herewith in its entirety.

### TECHNOLOGICAL FIELD

This present disclosure relates to the detection, and optionally the monitoring, of macrocyclic lactone resistance in nematodes based on the characterization of the Dyf7 gene (or gene ortholog) and its corresponding gene products.

### BACKGROUND

Macrocyclic lactone endectocides, which include ivermectin (IVM), abamectin, doramectin, eprinomectin, selamectin, moxidectin (MOX) and milbemycin oxime, are the most important and most widely used anthelmintics for the control of parasitic nematodes in animals, including, cattle, sheep, goats, horses, cats and dogs, swine, deer, alpaca and are also important for the control of parasitic nematodes in humans. They can also be used to control plant parasitic nematodes. Their widespread use has been due to their very high potency, safety and ease of administration. They can be administered, depending on the target species, orally, by injection e.g., subcutaneously, and by dermal application. They can also be applied topically to plants. Unfortunately, there are increasing reports of resistance to the macrocyclic lactones (MLs) in animal and human parasitic nematodes. This is of great importance because there are few alternative anthelmintics and other classes of anthelmintic do not share all of the attributes of the macrocyclic lactones. Nevertheless, when resistance develops and the effectiveness of the macrocyclic lactone anthelmintics becomes reduced, it is important to be able to detect that resistance quickly and efficiently so that alternative control strategies can be considered to replace reliance on macrocyclic lactone anthelmintics. When resistance arises and macrocyclic lactones fail to control parasites there can be significant economic costs due to reduced live weight gain reduced milk and wool production and lower fertility rates in animals and the uncontrolled parasites can cause severe morbidity and death. In addition, inadequate parasite control is of concern for animal welfare and crop production.

Sometimes resistance is only suspected when animals begin to die or fail to grow as expected. Even then, it is not a simple matter, nor an inexpensive exercise, to confirm that the cause of these economic losses is the development of macrocyclic lactone endectocide resistance. The most commonly used method for determining anthelmintic resistance in animals is to conduct a type of efficacy test in vivo. Most commonly this will involve obtaining fecal samples from animals (usually a minimum of 10 animals is needed to obtain statistical significance) before macrocyclic lactone treatment and then again on the same animals (or paired treatment naïve control and treated animals) 10 to 18 days after treatment. Nematode egg counts are conducted on these fecal samples and the reduction in the fecal (nematode) egg count (fecal egg count reduction test or FECRT) is estimated for the mean of the group of animals. This involves considerable work, with the animals normally having to be handled at least twice, a person skilled in detecting nematode eggs by microscopy, and the results of the FECRT are only known after the animals have been treated (with the possible loss of the effectiveness of the pharmaceutical and possible production losses). Furthermore, the FECRT is insensitive for detecting anthelmintic resistance unless the level of resistance is already at 25% in the parasite population, and by itself, does not usually indicate which species of parasite is causing the resistance. In order to determine which species is causing the resistance a second series of assays, involving culturing nematode eggs in feces before and after the anthelmintic treatment and differentiating, microscopically, the larvae which develop from the eggs. These cultures require fecal sampling, incubation of the fecal samples for approximately 1 week and highly skilled personnel who can identify the larval stages of different species of nematodes.

Another in vivo test is the control test which involves the treatment of a group of animals and the post-mortem comparison of the treated group (again, approximately 10 animals/group) with the post-mortem examination of an untreated control group of animals. This control test is not done routinely because it is very expensive, time consuming, raises concerns for animal welfare, and requires highly skilled personnel who can conduct the test and identify and count parasites that survive the treatment.

To attempt to measure macrocyclic lactone resistance a number of in vitro biological assays have been developed which involve the exposure of larval stages of nematodes to macrocyclic lactone molecules, such as ivermectin, eprinomectin and abamectin. These in vitro assays include the larval development assay (LDA), the larval motility assay (LMA), and the larval feeding assay and rely on the ability of macrocyclic lactones to prevent the development of larval stages (usually the L3 larval stage is counted), to inhibit the motility of the larvae (usually the L3 larvae), and/or to inhibit pharyngeal pumping and feeding of the larval stages (L1 or L2 larvae). These biological assays for detecting macrocyclic lactone resistance require a high level of technical knowledge and competence, are relatively insensitive for detecting low level resistance, and are expensive. And so they are not widely used routinely to monitor for developing resistance, but rather tend to be only used to confirm resistance once high level resistance is suspected.

Anthelmintic resistance has been defined as being present when there is a greater frequency of individuals within a population able to tolerate doses of a compound than in a normal population of the same species and is heritable. By definition anthelmintic resistance involves a loss of efficacy to a given dose rate (usually the recommended dose rate) of the pharmaceutical and it has a genetic basis (is heritable). Having a genetic basis, it is likely that the resistance is due to the selection for certain DNA sequence(s), which is/are initially rare in a susceptible population, but with repeated treatment with the control agent (anthelmintic) increase in frequency as the more susceptible parasites are either killed, or have their reproduction and fitness reduced by the treatment, while that genetically resistant individuals survive and/or have a fitness advantage in the presence of the selective agent.

It would be highly desirable to be provided with an in vitro method and tools for detecting/monitoring macrocyclic lactone resistance in nematodes. Preferably, the methods would not rely on the recuperation of living nematodes from infected subjects. Even more preferably, this method could be designed to detect low to moderate levels of resistance quickly, inexpensively and with high sensitivity.

### BRIEF SUMMARY

One aim of the present disclosure is to provide methods for determining macrocyclic lactone resistance in nematodes based on the characterization of the Dyf-7 gene or its ortholog. It is shown herein that genetic polymorphisms located in the Dyf-7 gene or its ortholog are associated with susceptibility/resistance to macrocyclic lactone. In some embodiments, the proposed methods can thus be based on the determination of the presence or absence of at least one polymorphic locus located in the Dyf-7 gene or its ortholog to assess macrocyclic lactone susceptibility/resistance. It is also shown herein that a reduced expression of the gene products associated with the Dyf-7 gene or its ortholog is also associated with susceptibility/resistance to macrocyclic lactone. In some embodiments, the proposed methods can thus be based on the determination of level of expression of the gene products associated with Dyf-7 gene or its ortholog to assess macrocyclic lactone susceptibility/resistance.

In a first aspect, the present disclosure provides a method for assessing the susceptibility of a nematode to a macrocyclic lactone. Broadly, the method comprises (a) providing a genomic DNA sample of the nematode comprising a Dyf-7 gene or a Dyf-7 gene ortholog; (b) determining the nucleic acid identity of at least one polymorphic locus identified in FIG. 9 in the genomic DNA (at positions identified by triangles and arrows); (c) associating the nucleic acid identity obtained in step (b) either to the nucleic acid identity of a corresponding at least one polymorphic locus of a resistant phenotype or with the nucleic acid identity of a corresponding at least one polymorphic locus of a susceptible phenotype; and (d) characterizing the nematode as being susceptible to the macrocyclic lactone when the nucleic acid identity of the at least one polymorphic locus is determined to be associated with the susceptible phenotype or resistant to the macrocyclic lactone when the nucleic acid identity of the at least one polymorphic locus is determined to be associated with the resistant phenotype. In an embodiment, the at least one polymorphic locus is listed in Table 3. In another embodiment, the macrocyclic lactone is ivermectin. In still another embodiment, the nematode is from a Trichostrongylidae family which includes, but is not limited to, a Haemonchus genus (Haemonchus contortus for example). In another embodiment, the nematode is an adult nematode, a larva or an egg. In still another embodiment, the method further comprising, prior to step (b), providing a nucleic acid synthetic copy of the genomic DNA comprising the Dyf-7 gene or the Dyf-7 gene ortholog and using the nucleic acid synthetic copy to determine the nucleic acid identity. In yet a further embodiment, the method further comprises, prior to step (b), amplifying a portion of the Dyf-7 gene or the Dyf-7 gene ortholog corresponding to the portion of the Dyf-7 gene amplified with the following pair of primers: TCTTTCCAGTGGACGAGGTGTCA (SEQ ID NO: 11) and AGAGGTCGTCCATCAGTGCTTCT (SEQ ID NO: 12) to provide the nucleic acid synthetic copy. In an embodiment, the at least one polymorphic locus comprises Hco-Dyf7-141, Hco-Dyf7-234 and/or Hco-Dyf7-438. In yet another embodiment, the method comprises determining the nucleic acid identity of at least three polymorphic loci. In an embodiment, the at least three polymorphic loci comprise at least one of Hco-Dyf7-141, Hco-Dyf7-234 or Hco-Dyf7-438. In another embodiment, the at least three polymorphic loci comprise at least two of Hco-Dyf7-141, Hco-Dyf7-234 and/or Hco-Dyf7-438. In yet a further embodiment, the at least three polymorphic loci comprises Hco-Dyf7-141, Hco-Dyf7-234 and Hco-Dyf7-438.

In a second aspect, the present disclosure provides a commercial package for the detection of macrocyclic lactone resistance in nematodes. The commercial package comprises means for determining the nucleic acid identity of at least one polymorphic locus identified in FIG. 9 (and optionally listed in Table 3) and instructions for characterizing the nematode based on the nucleic acid identity.

In a third aspect, the present disclosure provides a method for assessing the susceptibility of a nematode to a macrocyclic lactone. Broadly, the method comprises (a) providing a gene product from a Dyf-7 gene or a Dyf-7 gene ortholog from the nematode; (b) determining the test level of the gene product; (c) comparing the test level of the gene product to a control level of the gene product of a Dyf-7 gene or a Dyf-7 gene ortholog associated with susceptibility to the macrocyclic lactone; and (d) characterizing the nematode as susceptible to the macrocyclic lactone when the test level is determined to be equal to or higher than the control level and as resistant to the macrocyclic lactone when the test level is determined to be lower than the control level. In an embodiment, the macrocyclic lactone is ivermectin. In another embodiment, the macrocyclic lactone is ivermectin. In still another embodiment, the nematode is from a Trichostrongylidae family which includes, but is not limited to, a Haemonchus genus (Haemonchus contortus for example). In still another embodiment, the nematode is an adult nematode, a larva or an egg. In yet another embodiment, the gene product is a transcript of the Dyf-7 gene or the Dyf-7 gene ortholog or a protein encoded by the Dyf-7 gene or the Dyf-7 gene ortholog.

Throughout the description of the present disclosure, several terms are used that are specific to the science of this field. For the sake of clarity and to avoid any misunderstanding, these definitions are provided to aid in the understanding of the specification and claims.

Allele. The term “allele” refers to one of a pair, or series, of forms of a genetic region that occur at a given locus in a chromosome. An “associated allele” refers to a specific allele at a polymorphic locus that is associated with a particular phenotype of interest, e.g., a predisposition to a disorder or a particular response to an agent. Within a population, given multiple loci, there may be more than one combination of alleles associated with a phenotype of interest.

Amplification. As used herein, the terms “amplification”, “amplifying” and the like refer generally to any process that results in an increase in the copy number of a (nucleic acid) molecule or set of related (nucleic acid) molecules. As it applies to polynucleotide molecules, amplification means the production of multiple copies of a polynucleotide molecule, or a portion of a polynucleotide molecule, typically starting from a small amount (usually undetectable without amplification) of a polynucleotide, until, typically, the amplified material becomes detectable by spectroscopic, photochemical, biochemical, immunochemical, chemical, or other detection. Amplification of polynucleotides encompasses a variety of chemical and enzymatic processes. The generation of multiple DNA copies from one or a few copies of a template DNA molecule during a polymerase chain reaction (PCR), a strand displacement amplification (SDA) reaction, a transcription mediated amplification (TMA) reaction, a nucleic acid sequence-based amplification (NASBA) reaction, or a ligase chain reaction (LCR) are forms of amplification. Amplification is not limited to the strict duplication of the starting molecule. For example, the generation of multiple cDNA molecules from a limited amount of RNA in a sample using RT-PCR is a form of amplification. Furthermore, the generation of multiple RNA molecules from a single DNA molecule during the process of transcription is also a form of amplification.

Antibody. As used herein, an “antibody” include monoclonal antibodies (including full length monoclonal antibodies), polyclonal antibodies, multispecific antibodies (e.g. bispecific antibodies), single domain antibodies and antibody fragments recognizing the same epitope as the complete version of the antibody. Examples of antibody fragments include, but are not limited to, Fab, Fab′, F(ab′)2, and Fv fragments; diabodies; linear antibodies; single-chain antibody molecules; and multi-specific antibodies formed from antibody fragments. The term “antibody” may also include chimeric or humanized antibodies.

Naturally occurring immunoglobulins have a common core structure in which two identical light chains (about 24 kD) and two identical heavy chains (about 55 or 70 kD) form a tetramer. The amino-terminal portion of each chain is known as the variable (V) region and can be distinguished from the more conserved constant (C) regions of the remainder of each chain. Within the variable region of the light chain is a C-terminal portion known as the J region. Within the variable region of the heavy chain, there is a D region in addition to the J region. Most of the amino acid sequence variation in immunoglobulins is confined to three separate locations in the V regions known as hypervariable regions or complementarity determining regions (CDRs) which are directly involved in antigen binding. Proceeding from the amino-terminus, these regions are designated CDR1, CDR2 and CDR3, respectively. The CDRs are held in place by more conserved framework regions (FRs). Proceeding from the amino-terminus, these regions are designated FR1, FR2, FR3, and FR4, respectively. The locations of CDR and FR regions and a numbering system have been defined by Kabat et al. (Kabat, E. A. et al., Sequences of Proteins of Immunological Interest, Fifth Edition, U.S. Department of Health and Human Services, U.S. Government Printing Office (1991)).

Antibody derivatives include, but are not limited to, humanized antibodies. As used herein, the term “humanized antibody” refers to an immunoglobulin that comprises both a region derived from a human antibody or immunoglobulin and a region derived from a non-human antibody or immunoglobulin. The action of humanizing an antibody consists in substituting a portion of a non-human antibody with a corresponding portion of a human antibody. For example, a humanized antibody as used herein could comprise a non-human variable region (such as a region derived from a murine antibody) capable of specifically recognizing a polypeptide encoded by the Dyf-7 gene (or its ortholog) as described herein and a human constant region derived from a human antibody. In another example, the humanized immunoglobulin can comprise a heavy chain and a light chain, wherein the light chain comprises a complementarity determining region derived from an antibody of non-human origin which binds to the polypeptide and a framework region derived from a light chain of human origin, and the heavy chain comprises a complementarity determining region derived from an antibody of non-human origin which binds to the polypeptide and a framework region derived from a heavy chain of human origin.

As used herein, the present disclosure also relates to fragments of the monoclonal antibodies. “Antibody fragments” comprise a portion of a full length antibody, generally the antigen binding or variable region thereof. As used herein, a “fragment” of an antibody (e.g. a monoclonal antibody) is a portion of an antibody that is capable of specifically recognizing the same epitope as the full version of the antibody. In the present disclosure, antibody fragments are capable of specifically recognizing the polypeptide. Antibody fragments include, but are not limited to, the antibody light chain, single chain antibodies, Fv, Fab, Fab′ and F(ab′)₂ fragments. Such fragments can be produced by enzymatic cleavage or by recombinant techniques. For instance, papain or pepsin cleavage can be used to generate Fab or F(ab′)₂ fragments, respectively. Antibodies can also be produced in a variety of truncated forms using antibody genes in which one or more stop codons have been introduced upstream of the natural stop site. For example, a chimeric gene encoding the heavy chain of an F(ab′)₂ fragment can be designed to include DNA sequences encoding the CH1 domain and hinge region of the heavy chain. Antibody fragments can also be humanized. For example, a humanized light chain comprising a light chain CDR (i.e. one or more CDRs) of non-human origin and a human light chain framework region. In another example, a humanized immunoglobulin heavy chain can comprise a heavy chain CDR (i.e., one or more CDRs) of non-human origin and a human heavy chain framework region. The CDRs can be derived from a non-human immunoglobulin.

Dyf-7 gene and Dyf-7 gene ortholog. The Dyf-7 gene encodes the DYF-7 protein which is a membrane protein responsible for neuronal extension in the amphids during larval development. DYF7's role in neuronal morphology also involves permeability of amphid and phasmid neurons to external dyes, chemotaxis to ammonium chloride, avoidance of high osmotic stimuli, male mating and dauer formation. In the context of the present disclosure, a “Dyf-7 gene ortholog” is understood to be a gene in a different species that evolved from a common ancestral gene by speciation. In the context of the present disclosure, a Dyf7 gene ortholog encodes a protein having the same biological function as the DYF-7 protein. In some embodiment, the Dyf-7 orthologs have at least 60%, at least 65%, at least 70%, at least 75%, at least 80%, at least 90%, at least 95%, at least 96%, at least 97%, at least 98% or at least 99% identity with the nucleic acid sequence (genomic or cDNA) of the Dyf-7 gene. Dyf-7 orthologs include, but are not limited to, the Dyf-8 gene (C. elegans). Table 1 below provide a list of Dyf7 gene orthologs.

TABLE 1
Orthologs of H. contortus Dyf7 gene and partial sequence homology available in other
nematodes.

| Nematode - (From NCBI | | Significant | of | |
|---|---|---|---|---|
| http://www.ncbi.nlm.nih.gov/) | Score | Value | % Identity | Accession number NCBI |
| Loa-loa hypothetical protein (LOAG 09931) | 417 | 1e−112 | 72 | XM_003145458.1 |
| mRNA complete CDS | | | | |
| Caenorhabditis briggsae CBR-DYF-8 protein | 208 | 9e−50 | 67 | XM_002643380.1 |
| (cbr-dyf-8) mRNA complete CDS | | | | |
| Caenorhabditis elegans DYF-7 protein (dyf-7) | 206 | 3e−49 | 67 | NM_077229.4 |
| mRNA complete CDS | | | | |
| Caenorhabditis remanei CRE-DYF7 protein | 206 | 3e−49 | 67 | XM_003105631.1 |
| (cre-dyf-7) mRNA complete CDS | | | | |
| Brugia malayi hypothetical protein partial | 215 | 9e−31 | 76 | XM_001894139.1 |
| mRNA | | | | |
| Onchocerca volvulus putative dyf gene, partial | 64.4 | 2e−06 | 78 | AY934537.1 |
| CDS | | | | |
| Caenorhabditis elegans Cosmid C43C3, | 112 | 1e−04 | 77 | Z47067.2 |
| complete sequence | | | | |
| Dirofilaria immitis nuclear genes assembly | 56 | 1e−06 | 84(61/72) | http://salmo.bio.ed.ac.uk/cgi-bin/gbrowse/gbrowse/nDi.2.2.2/ |
| http://salmo.bio.ed.ac.uk/cgi- | | | | |
| bin/gbrowse/gbrowse/nDi.2.2.2/ | | | | |
| Ascaris suum ASCF_6009_1429 31 1305 | 2147 | 2.8e−92 | 69(713/1027) | http://salmo.bio.ed.ac.uk/fgb2/gbrowse/ |
| ascaris/?name=id:75782;dbid=general | | | | |
| 94abnormal_DYe_Filling_family_member_(dyf- | | | | |
| 8) | | | | |

Identity. The term “identity”, as known in the art, refers to a relationship between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. Identity and similarity can be readily calculated by known methods, including but not limited to those described in A. M. Lesk (ed), 1988, Computational Molecular Biology, Oxford University Press, NY; D. W. Smith (ed), 1993, Biocomputing. Informatics and Genome Projects, Academic Press, NY; A. M. Griffin and H. G. Griffin, H. G (eds), 1994, ComputerAnalysis of Sequence Data, Part 1, Humana Press, NJ; G. von Heinje, 1987, Sequence Analysis in Molecular Biology, Academic Press; and M. Gribskov and J. Devereux (eds), 1991, Sequence Analysis Primer, M Stockton Press, NY; H. Carillo and D. Lipman, 1988, SIAM J. Applied Math., 48:1073.

A nucleic acid or fragment thereof is “substantially identical” or “substantially homologous” to another if, when optimally aligned (with appropriate nucleotide insertions and/or deletions) with the other nucleic acid (or its complementary strand), there is nucleotide sequence identity in at least 60% of the nucleotide bases, usually at least 70%, more usually at least 80%, preferably at least 90%, and more preferably at least 95 to 98% of the nucleotide bases. Alternatively, substantial homology or substantial identity exists when a nucleic acid or fragment thereof will hybridize, under selective hybridization conditions, to another nucleic acid (or a complementary strand thereof). Selectivity of hybridization exists when hybridization which is substantially more selective than total lack of specificity occurs. Typically, selective hybridization will occur when there is at least about 55% sequence identity over a stretch of at least about nine or more nucleotides, preferably at least about 65%, more preferably at least about 75%, and most preferably at least about 90%. The length of homology or identity comparison, as described, may be over longer stretches, and in certain embodiments will often be over a stretch of at least 5 nucleotides, at least 14 nucleotides, at least 20 nucleotides, more usually at least 24 nucleotides, typically at least 28 nucleotides, more typically at least 32 nucleotides, and preferably at least 36 or more nucleotides.

Gene product. In the context of the present disclosure, a “gene product” refers to a molecule which is transcribed from the Dyf-7 gene (or the Dyf-7 gene ortholog) (also referred to as a Dyf-7 gene transcript or a Dyf-7 gene ortholog transcript) as well as a molecule which is translated from the Dyf-7 gene transcript (or the Dyf-7 gene ortholog transcript) (also referred to as a DYF-7 protein).

Indel. In the context of the present disclosure, the term indel refers to a nucleic acid molecule which may be present (e.g. inserted) or absent (e.g. deleted) when the nucleotide sequence of the Dyf-7 gene (or gene ortholog) of a susceptible strain is compared to the nucleotide sequence of the Dyf-7 gene (or gene ortholog) of a resistant strain. FIG. 9 does provide the nature and location of indels which have been identified when the sequence of a nucleic acid molecule associated to the Dyf-7 gene (or gene ortholog) of a susceptible stain (PF23-24) was compared to a resistant strain (MOF23-9). Said indels are located at position 1445 in the intron between exons 1 and 2; at positions 1799 and 1819 in the intron between exons 2 and 3; at positions 2092 to 2095 in the intron between exons 3 and 4; at position 2488 in the intron between exons 5 and 6; at positions 2911 to 2912, 2955 to 2958, 2998 to 3008, 3225 and 3306 in the intron between exons 7 and 8; and at positions 3681, 3748 to 3785, 3819 to 3861, 3872 to 3924, 3962 to 4022, 4052 to 4053, 4165 to 4275, 4325 to 4327, 4373, and 4389 in the intron between exons 8 and 9.

Macrocyclic lactone. Macrocyclic lactones (also referred to as MLs) are endectocides, which include, but are not limited to, ivermectin (IVM), abamectin, doramectin, eprinomectin, selamectin, moxidectin (MOX) and milbemycin oxime. This class of anti-helminthics is used in human as well as in veterinary medicine.

Nucleic acid. As used herein, “nucleic acid”, “nucleotide sequence” or “nucleic acid molecule” refer to a polymer of DNA and/or RNA which may be single or double stranded and optionally containing synthetic, non-natural or altered nucleotide bases capable of incorporation into DNA or RNA polymers. “Nucleic acid”, “nucleotide sequence” or “nucleic acid molecule” may encompass genes, cDNA, DNA (e.g. genomic DNA) and RNA encoded by a gene. Nucleic acids or nucleic acid sequences may comprise at least 3, at least 10, at least 100, at least 1 000, at least 5 000, or at least 10 000 nucleotides or base pairs. “Nucleic acid”, “nucleotide sequence” or “nucleic acid molecule” may be modified by any chemical and/or biological means known in the art including, but not limited to, reaction with any known chemicals such as alkylating agents, browning sugars, etc; conjugation to a linking group; methylation; oxidation; ionizing radiation; or the action of chemical carcinogens. Such nucleic acid modifications may occur during synthesis or processing or following treatment with chemical reagents known in the art. Probes, oligonucleotides and primers can be made from nucleic acid bases or modified nucleic acid bases. As used herein, a nucleic acid molecule (such as a probe, an oligonucleotide and/or a primer) “consists essentially of” or “consisting essentially of” means that the nucleic acid sequence may include one or more nucleotide bases, including within the sequence or at one or both ends of the sequence, but that the additional nucleotide bases do not materially affect the function of the nucleic acid sequence.

An “isolated nucleic acid molecule” may refer to a nucleic acid molecule that does not occur in nature as part of a larger polynucleotide sequence; and/or may be substantially free from any other nucleic acid molecules or other contaminants that are found in its natural environment. As used herein, an “isolated nucleic acid molecule” may also encompass recombinant or synthetically produced nucleic acid molecules. For example, a synthetic copy of a genomic DNA sequence is considered to be an isolated nucleic acid molecule.

Nucleic acids also specifically includes a molecule or a collection of molecules isolated from the genome of the nematode. Such molecules are collectively referred to as the genomic DNA of the nematode.

Peptide. As used herein, the terms “peptide”, “oligopeptide”, “polypeptide” and “protein” may be used interchangeably and encompasses any chain of naturally or non-naturally occurring amino acids (either D- or L-amino acids), regardless of length (e.g. at least 5, 6, 8, 10, 12, 14, 16, 18, 20, 25, 30, 40, 50, 100 or more amino acids) or post-translational modification (e.g., glycosylation or phosphorylation) or the presence of e.g. one or more non-amino acyl groups (for example, sugar, lipid, etc.) covalently linked to the peptide, and includes, for example, natural proteins, synthetic or recombinant polypeptides and peptides, hybrid molecules, peptoids, peptidomimetics, etc. Peptides may also be monomeric or multimeric. Peptide fragments may comprise a contiguous span of at least 5, at least 10, at least 25, at least 50, at least 100, at least 250, at least 500, at least 1000, at least 1500, or at least 2500 consecutive amino acids and may retain the desired activity of the full length peptide.

Peptide mimetics. Peptide mimetics mimic the three-dimensional structure of a polypeptide. Such peptide mimetics may have significant advantages over naturally occurring peptides, including, for example: more economical production, greater chemical stability, enhanced pharmacological properties (half-life, absorption, potency, efficacy, etc.), altered specificity (e.g., a broad-spectrum of biological activities), reduced antigenicity and others. In one form, mimetics are peptide-containing molecules that mimic elements of protein secondary structure. The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule.

Polymerase chain reaction. As used herein, the term “polymerase chain reaction” (PCR) refers to a method for amplification well known in the art for increasing the concentration of a segment of a target polynucleotide in a sample, where the sample can be a single polynucleotide species, or multiple polynucleotides. Generally, the PCR process consists of introducing a molar excess of two or more extendable oligonucleotide primers to a reaction mixture comprising the desired target sequence(s), where the primers are complementary to opposite strands of the double stranded target sequence. The use of the primers enable the production of amplicons that represent a target or standard sequence. The reaction mixture is usually subjected to a program of thermal cycling in the presence of a DNA polymerase, resulting in the amplification of the desired target sequence flanked by the DNA primers. Reverse transcriptase PCR (RT-PCR) is a PCR reaction that uses an RNA template and a reverse transcriptase, or an enzyme having reverse transcriptase activity, to first generate a single stranded DNA molecule prior to the multiple cycles of DNA-dependent DNA polymerase primer elongation. Multiplex PCR refers to PCR reactions that produce multiple copies of more than one product or amplicon in a single reaction, typically by the inclusion of more than two different primers in a single reaction.

Polymorphic loci. In the context of the present disclosure, a polymorphic locus corresponds to a location along a nucleic acid molecule where a genetic polymorphism is present. The polymorphism may be, for example, an indel and/or a SNP. The polymorphism may also be a combination of indels and/or SNPs. FIG. 9 does provide the nature and location of polymorphic loci which have been identified when the sequence of a nucleic acid molecule associated to the Dyf-7 gene (or gene ortholog) of a susceptible stain (PF23-24) was compared to a resistant strain (MOF23-9).

Resistance/Susceptibility to macrocyclic lactones. As used in the context of the present disclosure, a nematode is said to be resistant to macrocyclic lactone if less than about 93%, less than about 91%, less than about 89%, less than about 87%, less than about 85%, less than about 83%, less than about 81%, less than about 79%, less than about 77%, less than about 75%, less than about 73%, less than about 71%, less than about 69%, less than about 67%, less than about 65%, less than about, 63%, less than about 61%, less than about 59%, less than about 57%, less than about 55%, less than about 53%, less than about 51%, less than about 49%, less than about 47%, less than about 45%, less than about 43%, less than about 41%, less than about 39%, less than about 37%, less than about 35%, less than about 33%, less than about 31%, less than about 29%, less than about 27%, less than about 25%, less than about 23%, less than about 21%, less than about 19%, less than about 17%, less than about 15%, less than about 13%, less than about 11%, less than about 9%, less than about 7%, less than about 5%, less than about 3%, less than about 1% or if 0% of nematodes die following exposure to a LD₉₅ dose or concentration of macrocyclic lactone. On the other hand, a nematode is said to be sensitive to a macrocyclic lactone if at most about 5%, at most about 4%, at most about 3%, at most about 2%, at most about 1% or if 0% of nematodes survive following exposure to a LD₉₅ dose or concentration of macrocyclic lactone.

Single Nucleotide Polymorphism. “Single nucleotide polymorphism” or “SNP” refer to a variation of a single nucleotide at a specific position within a given population. This includes the replacement of one nucleotide by one or more nucleotide as well as the deletion or insertion of one or more nucleotide. Typically, SNPs are biallelic markers although tri- and tetra-allelic markers also exist. For a combination of SNPs, the term “haplotype” is used, e.g. the genotype of the SNPs in a single DNA strand that are linked to one another. In certain embodiments, the term “haplotype” is used to describe a combination of SNP alleles, e.g., the alleles of the SNPs found together on a single DNA molecule. In specific embodiments, the SNPs in a haplotype are in linkage disequilibrium with one another. FIG. 9 does provide the nature and location of SNPs which have been identified when the sequence of a nucleic acid molecule associated to the Dyf-7 gene (or gene ortholog) of a susceptible strain (PF23-24) was compared to a resistant strain (MOF23-9). Said SNPs are located at position 1549 in the intron between exons 1 and 2; at positions 1769 (SNP 141 in Table 3), 1781 (SNP 153 in Table 3) in exon 2; at positions 1923 (SNP 192 in Table 3), 1950, 1965 (SNP 234 and 246 respectively in Table 3), 1977 (SNP 267 in Table 3), 1998 (SNP 288 in Table 3) in exon 3; at positions 2126 (SNP 303 in Table 3), 2141 (SNP 309 in Table 3), 2147 (SNP 312 in Table 3), 2150 (SNP 396 in Table 3), 2196, 2234 (SNP 402 and 438 respectively in Table 3) and 2240 (SNP 447 in Table 3) in exon 4; at positions 2341 (SNP 462 in Table 3), 2350, 2365, 2389, 2398, 2422, 2440 and 2449 in exon 5; at positions 2477, 2487, 2490, 2502, 2503, 2505, 2508, 2509, 2510 and 2540 in the intron between exons 5 and 6; at positions 2563, 2572, 2626 in exon 6; at positions 2657, 2661, 2664, 2666, 2672 to 2676, 2678, 2680 to 2686, and 2688 to 2691 in the intron between exons 6 and 7; at positions 2715, 2742, 2754, 2787, 2835 and 2859 in exon 7; at positions 2871 to 2874, 2877 to 2879, 2881, 2890, 2892, 2896 to 2897, 2901 to 2902, 2905, 2915, 2918, 2920, 2922, 2928, 2930, 2941, 2947, 2951 to 2952, 2967, 2975 to 2976, 2982, 2988 to 2989, 2994, 2996 to 2998, 3011 to 3012, 3014, 3016, 3020 to 3023, 3036, 3041, 3045, 3047 to 3048, 3050, 3052 to 3053, 3062, 3064, 3073, 3075, 3077 to 3079, 3082, 3084 to 3086, 3089, 3091, 3094, 3099, 3106, 3108, 3110, 3114, 3117, 3121, 3124, 3126, 3129, 3134, 3144, 3150, 3155, 3160 to 3161, 3165, 3177 to 3178, 3180, 3183, 3202 to 3203, 3206, 3210, 3218, 3227, 3229, 3232, 3238, 3240 to 3241, 3248, 3254, 3259, 3262, 3264, 3266, 3272 to 3275, 3284 to 3287, 3293, 3301, 3303, 3308, 3355, 3378 and 3391 in the intron between exons 7 and 8; and at positions 3577, 3589 to 3590, 3599, 3612, 3632, 3632, 3641, 3649, 3672, 3680, 3685, 3688, 3691, 3693 to 3695, 3697, 3699, 3706, 3709, 3719, 3722 to 3723, 3729, 3740, 3742 to 3744, 3746 to 3747, 3787 to 3788, 3794, 3797, 3801 to 3802, 3805 to 3806, 3808 to 3813, 3815 to 3816, 3863, 3870 to 3871, 3926, 3931, 3934 to 3935, 3938, 3941, 3945 to 3946, 3952, 3957, 3960 to 3961, 4024, 4027, 4030 to 4031, 4034, 4036, 4038 to 4041, 4044, 4048 to 4050, 4056 to 4058, 4062, 4071, 4073, 4077, 4079, 4083, 4085 to 4089, 4091, 4093 to 4101, 4106 to 4107, 4110, 4112 to 4113, 4115, 4119, 4124, 4127, 4129 to 4133, 4137, 4148, 4151, 4155, 4164, 4279 to 4281, 4283, 4291, 4293, 4295, 4299, 4301, 4303 to 4304, 4307 to 4309, 4313, 4315 to 4318, 4320 to 4322, 4324, 4331, 4333 to 4334, 4339 to 4342, 4344 to 4345, 4350 to 4351, 4354, 4359, 4365 to 4367, 4378, 4381, and 4384 to 4385 in the intron between exons 8 and 9.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 provides the sequences alignment of the Dyf7 amplified fragment between the susceptible strain (Bristol N2, 1^{st} line, SEQ ID NO: 29) and the resistant strains (IVR6 and IVR10, 2^{nd} and 3^{rd} line respectively, SEQ ID NO: 30 and 31 respectively). A CT-deletion (corresponding to positions 9700521 and 9700522 on the chromosome X in the whole C. elegans genome) was only detected in the resistant strains. Consensus sequence (SEQ ID NO: 32) is provided on the 4^{th} line.

FIG. 2 illustrates the rescue of Dyf7 gene in C. elegans IVR10 strain. Effect of Dyf7 rescued (strains Dyf7 ttx 1 and Dyf7 ttx 2) on ivermectin sensitivity. Strains “ttx ctrl 1” and “ttx ctrl 2” contain only the ttx-3:GFP transgenic marker. Results are shown as the percentage of growth of transgenic nematodes (as measured by GFP expression) in function of the type of transgenic nematode and treatment. Light grey bars show strains grown in absence of ivermectin (0 ng/mL) and dark grey bars shows strains grown in presence of ivermectin (6 ng/ml).

FIG. 3 illustrates the predictive Dyf7 gene sequence in H. contortus annotated using the homologous C. elegans's Dyf7 gene as a reference to determine the open reading frame of Dyf7 gene in H. contortus. Hco-Dyf7 gene contains 10 exons and 9 introns. The neighboring genes of Dyf7 are npr-18 and F15G9.1, the orthologous genes in C. elegans.

FIG. 4 illustrates the PF23-24 (1^{st} line (SEQ ID NO: 33) and 3^{rd} line (SEQ ID NO: 35)) and MOF23-9 (2^{nd} line (SEQ ID NO: 34) and 4^{th} line (SEQ ID NO: 36)) alignment at Dyf7-4 in H. contortus showing the 17 SNP loci that can be used as molecular markers (identified with a lower case on the consensus line (SEQ ID NO: 37)) to discriminate between susceptible and resistant isolates. Arrows indicate exons 2, 3, 4 and 5.

FIG. 5 illustrates the quantitative real-time qRT-PCR analysis of Dyf7 gene expression in PF23 (susceptible, 1^{st} bar) and MOF23 (resistant, 2^{nd} bar) strains. Results are expressed in fold changes when compared to the PF23 strain. Error bars indicate the standard deviation from mean.

FIG. 6 provides the frequency histogram of the FIS index across MOF23 (resistant, light gray bars) and IVF23 (resistant, dark gray bars) populations for the 106 SNP loci.

FIG. 7 provides the frequency histogram of the Weir and Cockerham's estimate of Wright's FST, across MOF23 and IVF23 populations for the 106 SNPs loci.

FIG. 8 provides the neighbor-joining dendogram of pairwise sequence alignments among H. contortus susceptible (PF23) and macrocyclic lactone resistant (IVF23 and MOF23) strains for the 106 SNP markers.

FIG. 9 provides the nucleic acid sequence alignment of the whole H. contortus Dyf-7 gene. The alignment contains two cDNA sequences (dyf7-PF23 (SEQ ID NO: 25) and dyf7-MOF23 (SEQ ID NO: 26)) and 2 genomic DNA (gDNA) sequences, (dyf7-PF23 (SEQ ID NO: 27) and dyf7-MOF23 (SEQ ID NO: 28)). The single nucleotide polymorphisms (SNPs) are label with “A” underline and the insertions/deletions (indel) are labeled with “→” underline.

### DETAILED DESCRIPTION

In accordance with the present disclosure, there is provided a method of detecting macrocyclic lactone resistance of nematodes as well as tools and commercial packages to perform the method. The method is based on the characterization of the Dyf-7 gene (or the Dyf-7 gene ortholog) and/or its associated gene products. As it is will discussed below, the wild-type expression level and/or stability of the Dyf-7 gene transcript, Dyf-7 gene ortholog transcript, a wild-type expression of the level and/or activity of the DYF-7 protein (or a corresponding protein encoded by the Dyf-7 ortholog) as well as the presence of specific polymorphisms in the Dyf-7 gene (or gene ortholog) are more frequently associated with nematodes being sensitive to macrocyclic lactones. As it will also be discussed below, a lower level expression level or stability of the Dyf-7 gene transcript, Dyf-7 gene ortholog transcript, a lower level of expression or activity of the DYF-7 protein (or a corresponding protein encoded by the Dyf-7 ortholog) as well as the presence of specific polymorphisms in the Dyf-7 gene (or gene ortholog) are more frequently associated with nematodes being resistant to macrocyclic lactones. Consequently, by characterizing the Dyf-7 gene (or Dyf-7 gene ortholog) and/or its associated products, it is possible to assess the likelihood of resistance to macrocyclic lactones in nematodes.

The methods described herein can be used for determining susceptibility/resistance to macrocyclic lactone of any nematode expressing the Dyf-7 gene or its ortholog. In order to do, a sample from the nematode (such as a genomic DNA sample or an associated gene product) is obtained. The sample can be obtained from an in vitro culture of the nematode. The sample can also be obtained from a biological sample of a subject at least suspected of being infected by the parasite. The subject may be, without limitation, livestock (such as, for example, cattle, sheeps, goats, horses, cats, dogs, swine, deer, alpaca) as well as humans. In the context of the present disclosure, a biological sample may be any sample (e.g. bodily fluid, blood, plasma, serum, cerebrospinal fluid, lymph, secretion, exudate, saliva, milk, stools, urine, epithelial cell swab, sweat, organ, tissue, etc) from the subject. In an embodiment, the sample is a stool sample containing at least one nematode egg. The nematodes of the biological sample can be optionally expanded in vitro prior to the isolation of the genomic DNA or its corresponding gene products. For example, the nematode egg can be expanded in vitro prior to the isolation of the genomic DNA by being cultured under conditions so as to allow at least one nematode larva to hatch.

Some of the methods described herein are based on the detection of alterations in the genomic DNA of the nematodes (and not transcripts or proteins). These methods are not limited to the characterization of adult nematodes. They can be successfully applied to nematodes at the larval stage (L1, L2, L3 or L4 stages) and even to a nematode egg.

The methods described herein can be practiced on nematodes of the Trichostrongylidae family. This family includes, but is not limited to, the genus Haemonchus (including the species Haemonchus contortus), the genus Teladorsagia (including the species Teladorsagia circumcincta), the genus Trichostrongylus (including the species Trichostrongylus colubriformis and Trichostongylus spp.), the genus Ostertagia (including the species Ostertagia ostertagi), the genus Cooperia (including the species Cooperia oncophora and Cooperia spp.) and the genus Nematodirus (including the species Nematodirus spp.).

The methods described herein can be used to determine the susceptibility/resistance phenotype of a nematode in view of an anti-helminthic macrocyclic lactone. The methods can be applied to determine the susceptibility/resistance phenotype towards a single macrocyclic lactone or a combination of macrocyclic lactones.

### Methods for Detecting Macrocyclic Lactone Resistance Based on the Characterization of Dyf-7 Gene Products

Initially, a nematode (or a sample thereof) comprising the Dyf-7 gene product (or Dyf-7 gene ortholog product) is contacted with an analyte-specific reagent (ASR) capable of detecting the presence/level/activity of the gene product. This contact can be made in a reaction under conditions favoring the interaction between the ASR and the gene product. The nematode is preferably provided as an adult nematode or as an adult nematode population. The nematode can be provided from an in vitro culture or directly from a biological sample of subject at least suspected of being infected by the nematode. In the assays described herein, the contact between the Dyf-7 gene product (or the Dyf-7 gene ortholog product) and the ASR can be made in a reaction vessel. The reaction vessel can be any type of container that can accommodate the measurement of a Dyf-7 gene product's (or a Dyf-7 gene ortholog product) parameter (through the use of the ASR).

Once the gene product has been contacted with the ASR, a measurement or value of a parameter of the gene product is made. This assessment may be made directly in the reaction vessel (by using a probe) or on a sample of such reaction vessel. The measurement of the parameter of the gene product (through the use of the ASR) can be made either at the RNA level and/or the polypeptide level.

The measuring step can rely, as an ASR, on the addition of a quantifier specific to the parameter to be assessed to the reaction vessel or a sample thereof. The quantifier can specifically bind to a parameter of the gene product that is being assessed, such as, for example, a gene product transcript (a probe or a primer for example) or a protein (an antibody for example). In those instances, the amount of the quantifier that specifically bound (or that did not bind) to the gene product can be determined to provide a measurement of the parameter of the gene product.

In an embodiment, the signal of the quantifier can be provided by a label that is either directly or indirectly linked to a quantifier. A label can be associated with radioactivity (¹²⁵I, ³⁵S, ¹⁴C, or ³H), either directly or indirectly, and the radioisotope detected by direct counting of radioemmission or by scintillation counting. Alternatively, the label can be an enzyme, for example, horseradish peroxidase, alkaline phosphatase, or luciferase, and the enzymatic label can be detected by determination of conversion of an appropriate substrate to product.

When the gene product is the DYF-7 protein, a parameter that is measured can be the polypeptide biological activity, the polypeptide quantity and/or stability. When the gene product is a nucleotide encoding a DYF-7 polypeptide or fragment thereof, the parameter can be the level of expression and/or stability of the DYF-7-encoding nucleotide. Even though a single parameter is required to enable the characterization of the individual or the agent, it is also provided that more than one parameter of the gene product may be measured and even that more than one ASRs may be used.

If the measurement of the parameter is performed at the nucleotide level, then the transcription activity of the promoter associated with the transcript gene product can be assessed. This assessment can be made, for example, by using a reporter vector. Such reporter vectors can include, but are not limited to, the promoter region of the Dyf-7 gene or its ortholog (or fragment thereof) being operably linked to a nucleotide encoding a reporter polypeptide (such as, for example, β-galactosidase, green-fluorescent protein, yellow-fluorescent protein, etc.). Alternatively or complementarily, the stability and/or the expression level of the transcripts of the gene product can be assessed by quantifying the amount of such transcripts (for example using qPCR or real-time PCR) or the stability of such transcripts. In a further assay format, the gene products can be characterized by hybridization.

If the measurement of the parameter is performed at the polypeptide level, an assessment of the expression/activity of the DYF-7 protein can be performed. In an embodiment, the level of expression can be measured by, for example, an antibody-based technique (such as an ELISA, flow cytometry, immunoprecipitation, gel-electrophoretic mobility assay, etc.), a micro-array, spectrometry, etc. In one embodiment, this assay is performed utilizing antibodies (or antibody products related thereto) specific to DYF-7.

It is also known that DYF-7 interacts with other proteins to mediate its activity. As such, it is possible to characterize the interaction between DYF-7 and its binding partners to assess DYF-7's biological activity. The interaction between two molecules can also be detected, e.g., using a fluorescence assay in which at least one molecule is fluorescently labeled. One example of such an assay includes fluorescence energy transfer (FET or FRET for fluorescence resonance energy transfer). A fluorophore label on the first “donor” molecule is selected such that its emitted fluorescent energy will be absorbed by a fluorescent label on a second “acceptor” molecule, which in turn is able to fluoresce due to the absorbed energy. Alternately, the “donor” protein molecule may simply utilize the natural fluorescent energy of tryptophan residues. Labels are chosen that emit different wavelengths of light, such that the “acceptor” molecule label may be differentiated from that of the “donor”. Since the efficiency of energy transfer between the labels is related to the distance separating the molecules, the spatial relationship between the molecules can be assessed. In a situation in which binding occurs between the molecules, the fluorescent emission of the “acceptor” molecule label in the assay should be maximal. A FET binding event can be conveniently measured through standard fluorometric detection means well known in the art (e.g., using a fluorimeter). Another example of a fluorescence assay is fluorescence polarization (FP). For FP, only one component needs to be labeled. A binding interaction is detected by a change in molecular size of the labeled component. The size change alters the tumbling rate of the component in solution and is detected as a change in FP. In another embodiment, the measuring step can rely on the use of real-time Biomolecular Interaction Analysis (BIA). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Once the measurement has been made, it is extracted and the value of the parameter of the gene product is compared to a control value. At this stage, it must be determined if the measured parameter of the gene product differs from a control value. In a first embodiment, the control value is associated with a susceptibility to macrocyclic lactones and as such, if the measured parameter is lower than the control value, then it is determined that the nematode is likely resistant to the macrocyclic lactone. Still in this embodiment, if the measured parameter is equal to or higher than the control value, then it is determined that the nematode is likely susceptible to the macrocyclic lactone. In a second embodiment, the control value is associated with a resistance to macrocyclic lactones and as such, if the measured parameter is equal to or lower than the control value, the nematode is determined to be more likely resistant to the macrocyclic lactone. Still in such embodiment, if the measured parameter is higher than the control value, then it is determined that the nematode is likely susceptible to the macrocyclic lactone.

In an embodiment, the comparison can be made by an individual. In another embodiment, the comparison can be made in a comparison module. Such comparison module may comprise a processor and a memory card to perform an application. The processor may access the memory to retrieve data. The processor may be any device that can perform operations on data. Examples are a central processing unit (CPU), a front-end processor, a microprocessor, a graphics processing unit (PPU/VPU), a physics processing unit (PPU), a digital signal processor and a network processor. The application is coupled to the processor and configured to determine the modulation of the level of the parameter with respect to the control value. An output of this comparison may be transmitted to a display device. The memory, accessible by the processor receives and stores data such as measured parameters of the gene product or any other information generated or used. The memory may be a main memory (such as a high speed Random Access Memory or RAM) or an auxiliary storage unit (such as a hard disk, a floppy disk or a magnetic tape drive). The memory may be any other type of memory (such as a Read-Only Memory or ROM) or optical storage media (such as a videodisc or a compact disc).

Once the comparison between the parameter of the gene product and the control value is made, then it is possible to characterize the phenotype of the nematode. This characterization is possible because, as shown herein, nematodes which are considered resistant to macrocyclic lactones express a lower level of Dyf-7 gene products (or Dyf-7 gene ortholog products) than nematodes which are considered susceptible to macrocyclic lactones.

The assays described herein can be applied to a single nematode or a populations of nematodes. The methods presented herein can also be used to monitor macrocyclic lactone resistance in a population of nematodes. The method can be practiced at a first point in time to determine, if any, the presence of macrocyclic lactone resistance in the population of nematodes. Then, the method can be performed at least at a second point in time, later in time than the first point in time, in order to determine if the phenotype of the population has changed.

The methods presented herein can also be used to determine if a subject intended to be treated with macrocyclic lactone or already being treated with macrocyclic lactone can benefit from a first or further macrocyclic lactone treatment. In order to do so, a biological sample from the subject suspected or known to be afflicted with a nematode infection is obtained either prior to macrocyclic lactone administration or after the administration of at least one dose of macrocyclic lactone. The gene products of the nematodes contained in the biological samples are isolated and analyzed to determine if the expression/level/stability/activity of the gene products are modulated. The absence of a decrease in the expression/level/stability/activity of the gene products (when compared to the expression/level/stability/activity associated to susceptible nematodes) indicates that the subject will benefit from a macrocyclic lactone treatment (because the infecting nematodes are considered susceptible to macrocyclic lactones). In an embodiment, the method can also encompass administering macrocyclic lactone in such subjects. On the other hand, a decrease in the expression/level/stability/activity of the gene products (when compared to the expression/level/stability/activity associated to susceptible nematodes) indicates that the subject will not benefit from a macrocyclic lactone treatment (because the infecting nematodes are considered resistant to macrocyclic lactone). In an embodiment, the method can also encompassing avoiding or discontinuing macrocyclic lactone treatment in such subject.

The methods presented herein can also be used to monitor the predisposition to macrocyclic lactone resistance in a treated subject. For example, the method can be practiced after the intake of at least a first dose of macrocyclic lactone by the treated subject to determine if the infecting nematodes are resistant or susceptible of developing a resistance against the macrocyclic lactone. In another example, the method can be practiced after the intake of a plurality of doses of macrocyclic lactone (and in some embodiment, during the entire period a macrocyclic lactone is administered to the infected subject) by the treated subject to determine if the infecting nematodes are resistant or susceptible of developing a resistance against the administered macrocyclic lactone.

The present disclosure also provides systems for performing the characterizations and methods described herein. These systems comprise a reaction vessel for placing the gene product sample, a processor in a computer system, a memory accessible by the processor and an application coupled to the processor. The application or group of applications is (are) configured for receiving a test value of a level of the gene product; comparing the test value to a control value and/or characterizing the phenotype of the nematodes in function of this comparison.

The present disclosure also provides a software product embodied on a computer readable medium. This software product comprises instructions for characterizing the phenotype of the nematodes according to the methods described herein. The software product comprises a receiving module for receiving a test value of a level of a gene product; a comparison module receiving input from the measuring module for determining if the test value is lower than, equal to or higher than a control value; a characterization module receiving input from the comparison module for performing the characterization based on the comparison.

In an embodiment, an application found in the computer system of the system is used in the comparison module. A measuring module extracts/receives information from the reaction vessel with respect to the expression/level/stability/activity of the gene product. The receiving module is coupled to a comparison module, which receives the value(s) of the level of the gene product and determines if this value is lower than, equal to or higher than a control value. The comparison module can be coupled to a characterization module. In another embodiment, an application found in the computer system of the system is used in the characterization module. The comparison module is coupled to the characterization module which receives the comparison and performs the characterization based on this comparison.

In a further embodiment, the receiving module, comparison module and characterization module are organized into a single discrete system. In another embodiment, each module is organized into different discrete systems. In still a further embodiment, at least two modules are organized into a single discrete system.

### Methods for Detecting Macrocyclic Lactone Resistance Based on the Characterization of Dyf-7 Gene

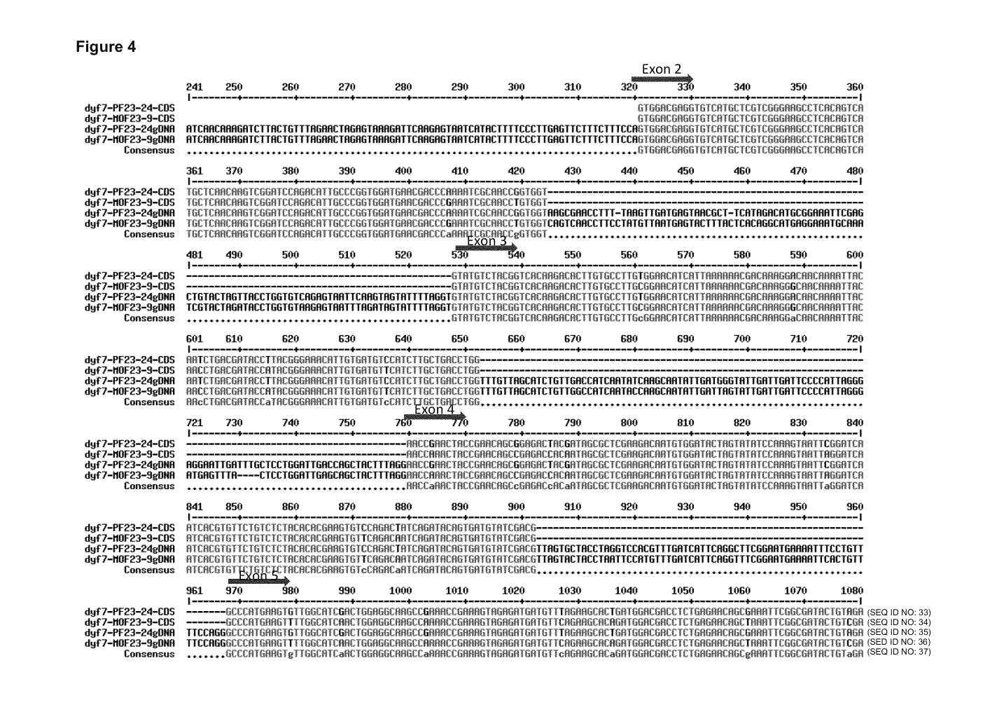

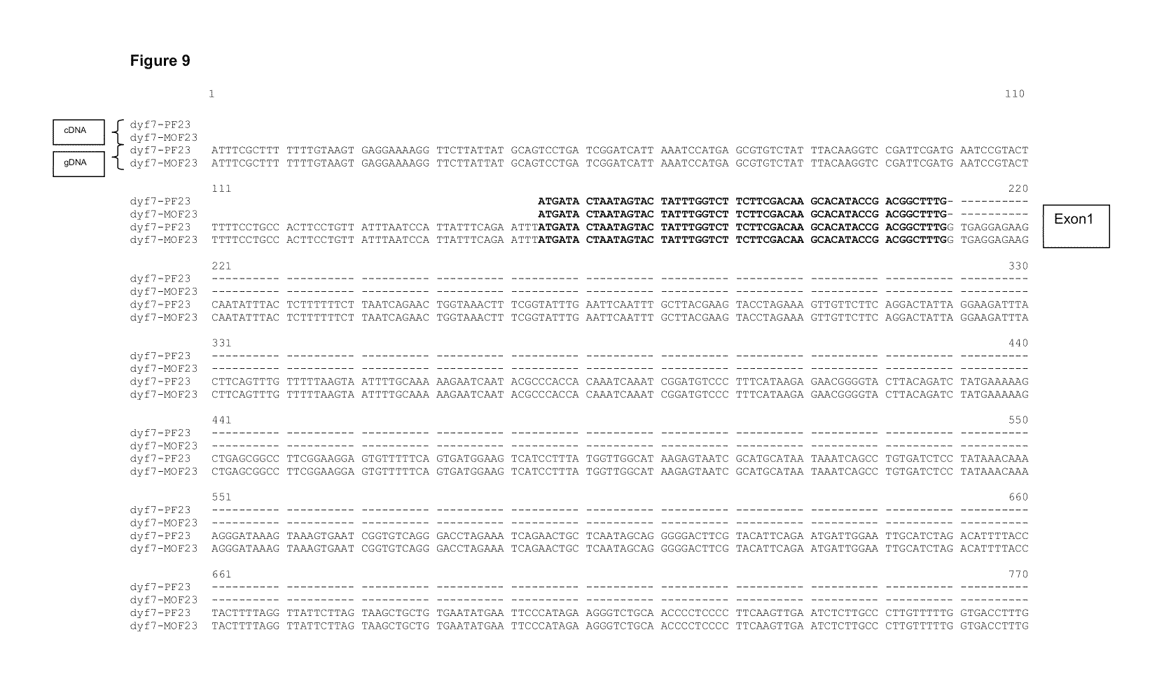

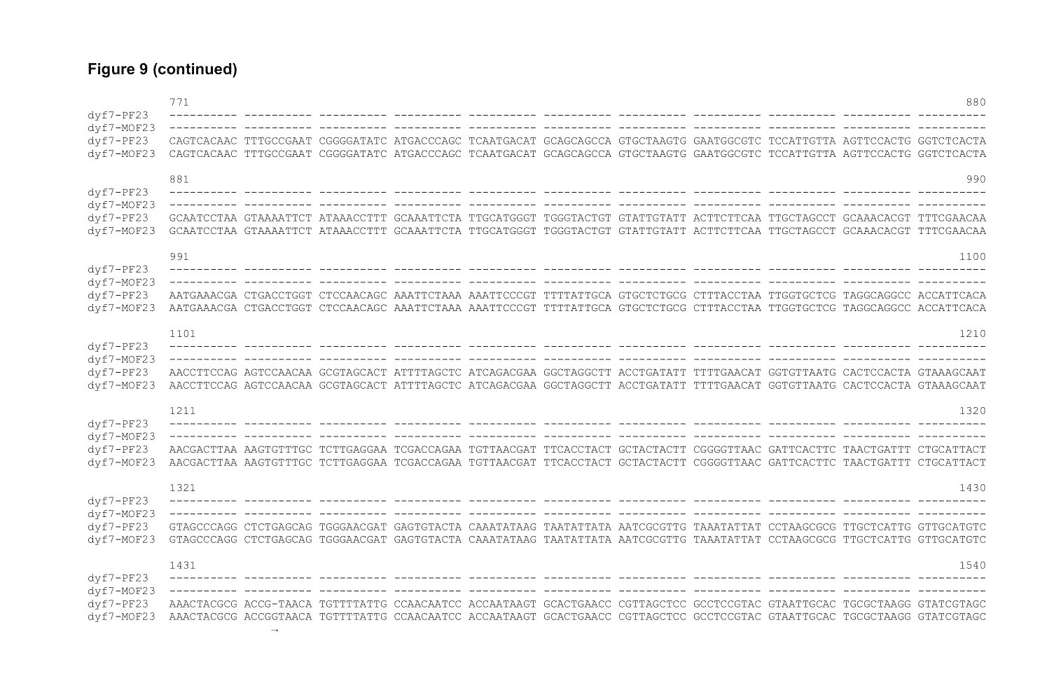

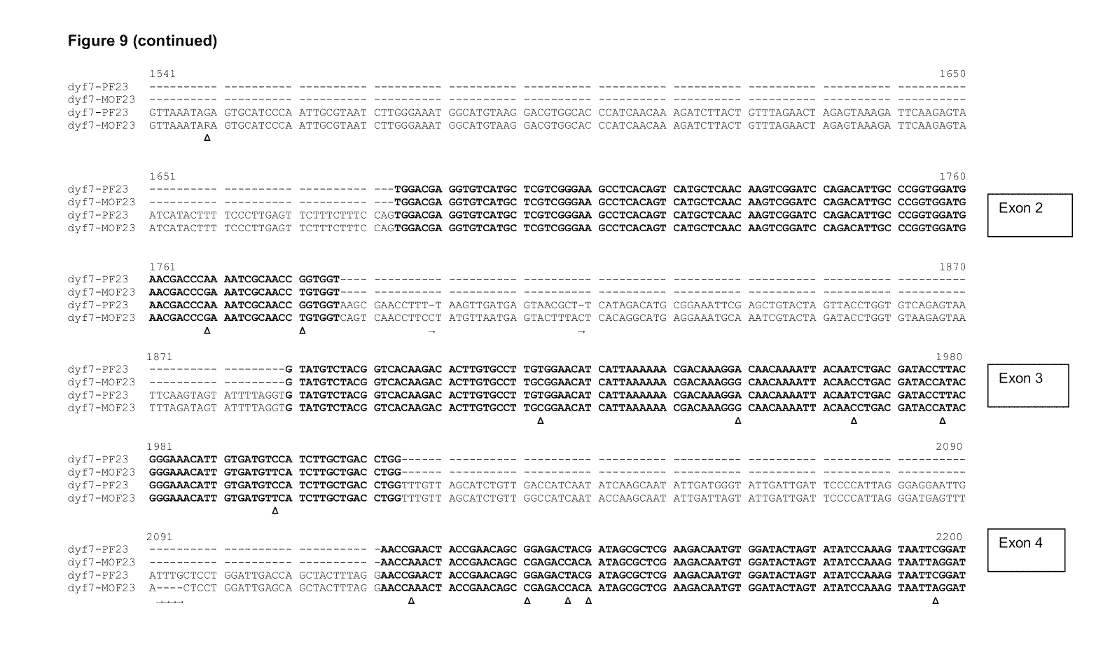

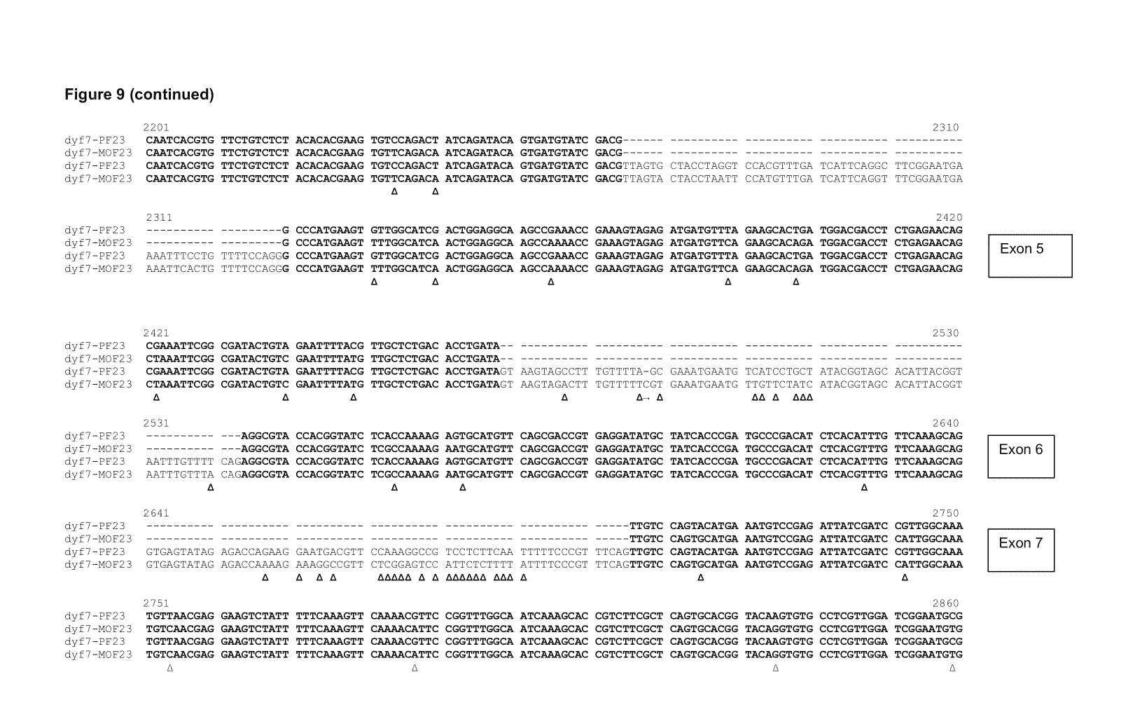

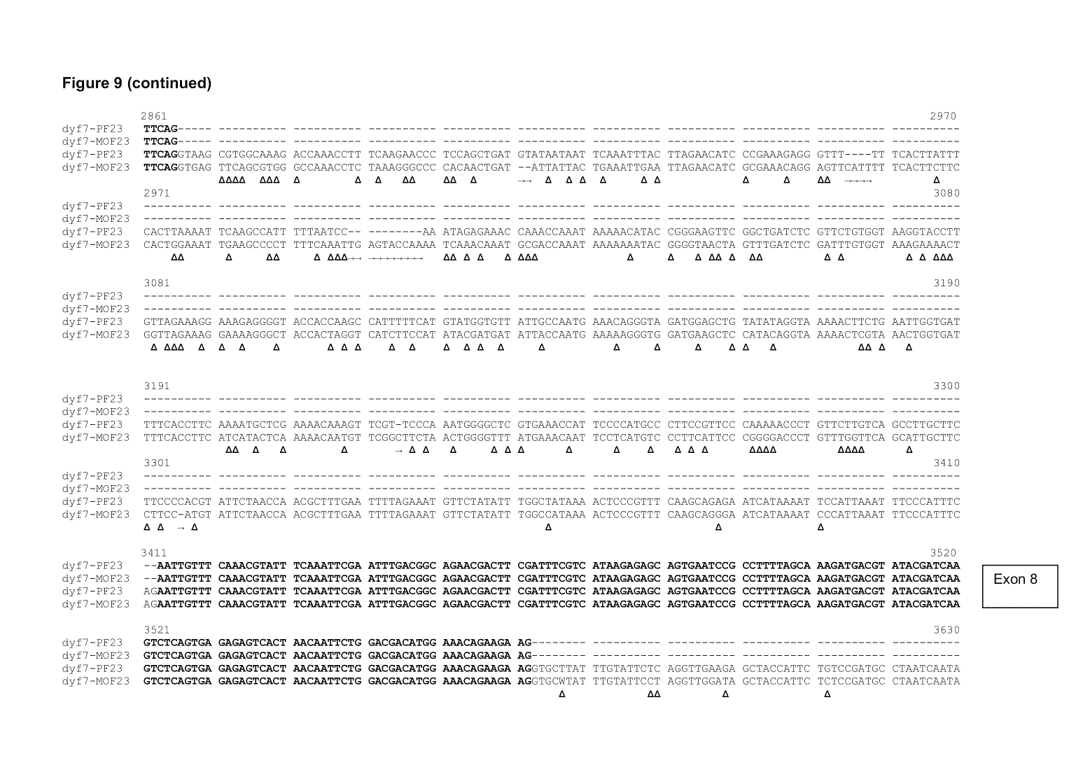

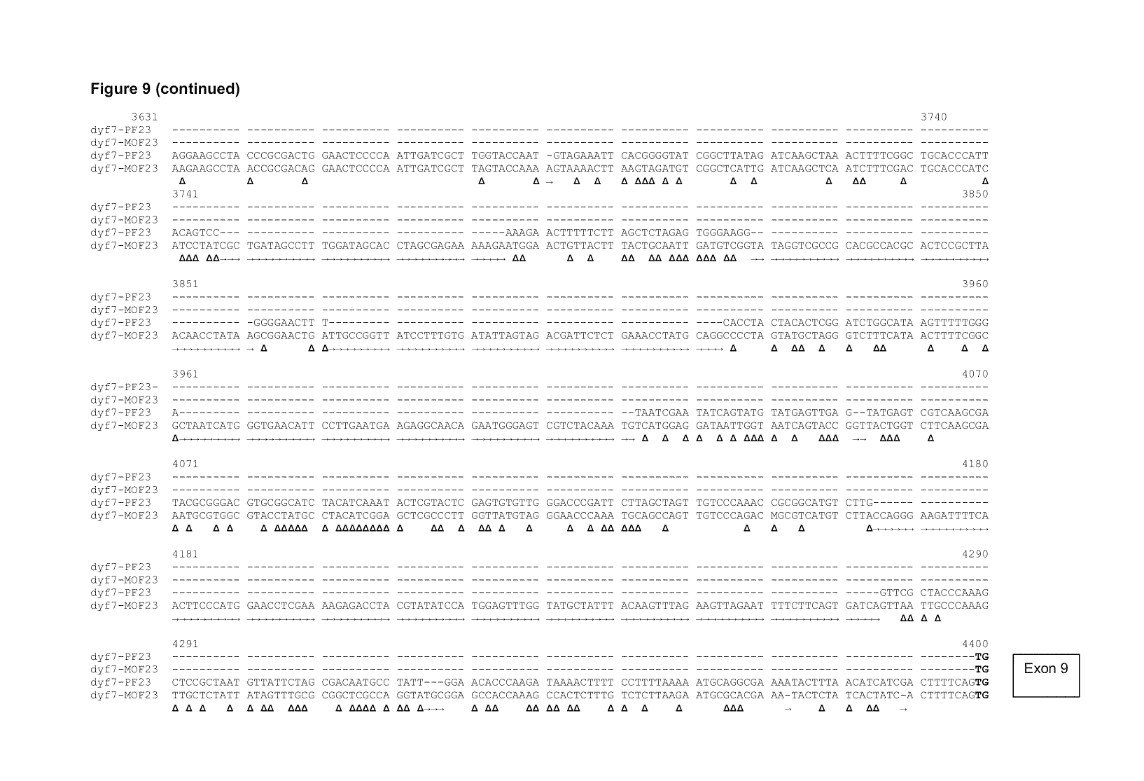

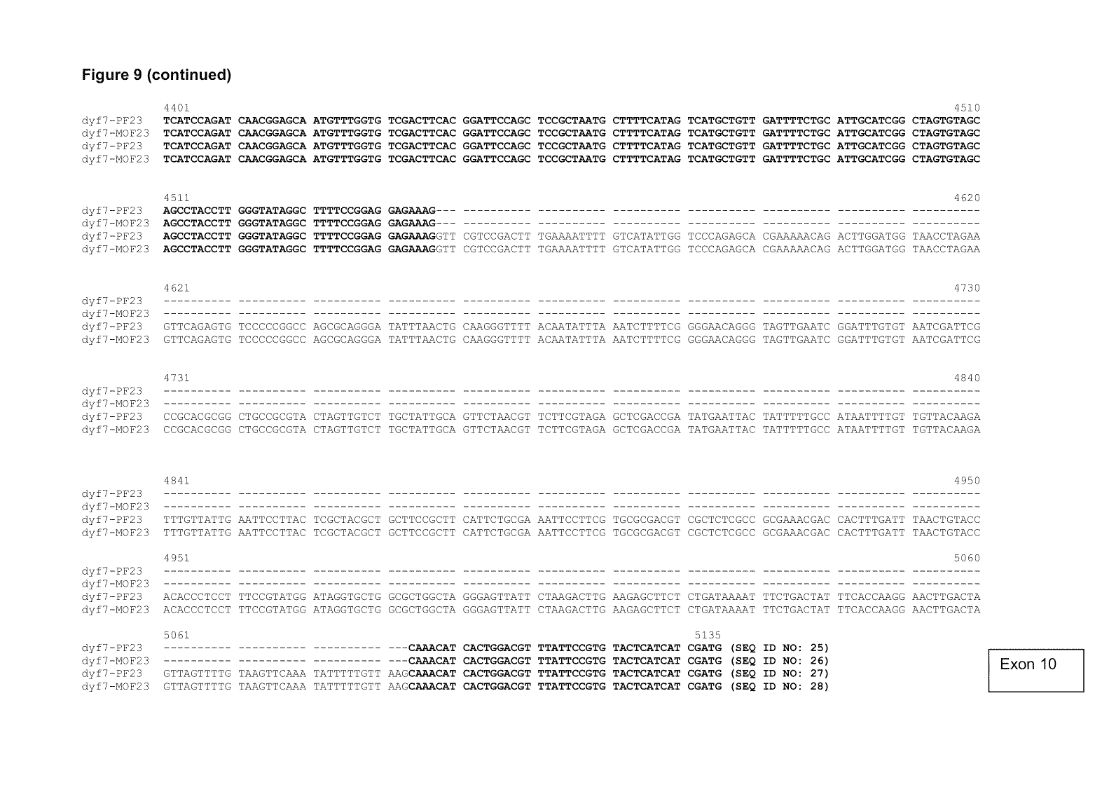

As indicated herein, the polymorphic loci identified in FIG. 9 (and, in some embodiments, presented in FIG. 4 or listed in Table 3 or Table 4 (which relates the SNP locations referred to in Table 3, FIG. 4 and FIG. 9)) are correlated to the phenotypes of the nematodes with respect to their susceptibility or resistance to macrocyclic lactones. Therefore, they provide an interesting tool for determining the phenotypes of the nematodes. They can also be valuable in determining the risk of a nematode (or a population of nematodes) more susceptible of developing macrocyclic lactone resistance as well as the usefulness of macrocyclic lactone treatment in infected subjects or subjects suspected of being infected.

The first step for determining susceptibility/resistance to macrocyclic lactone is to obtain a genomic DNA sample from a nematode. The genomic DNA can be obtained from an in vitro culture of the nematode. The genomic DNA can also be obtained from a biological sample of a subject at least suspected of being infected by the parasite. The nematodes of the biological sample can be optionally expanded in vitro prior to the isolation of the genomic DNA.

Once the genomic DNA has been obtained, and optionally copied or amplified, a genetic profile is determined. A genetic profile comprises genetic information including the nucleic acid identity of at least one polymorphic loci of the Dyf-7 gene (or its ortholog) identified in FIG. 9. In some embodiment, the genetic profile comprises one or more polymorphic locus listed in Table 3 and/or FIG. 4. In a further embodiment, the genetic profile comprises on or more polymorphic locus located within the segment 4 of the Dyf-7 gene (or a corresponding segment in the Dyf-7 gene ortholog). For example, the genetic profile can comprise the nucleic acid identity of at least one polymorphic locus identified in FIG. 9 (and optionally listed in Table 3). The genetic profile can also comprise a combination of genetic markers. In some embodiment, the genetic profile comprises the nucleic acid identity of at least two, three, four, five, six, seven, eight, nine, ten, eleven, twelve, thirteen, fourteen or fifteen polymorphic loci identified in FIG. 9 (and optionally listed in Table 3). In a preferred embodiment, the genetic profile comprises the nucleic acid identity of at least three polymorphic loci identified in FIG. 9 (and optionally listed in Table 3). The genetic profile can comprise, for example, at least one, at least two or at least three following polymorphic loci Hco-Dyf7-141, Hco-Dyf7-234 and/or Hco-Dyf7-438. In an alternate embodiment, besides the at least three polymorphic loci identified above, the genetic profile can comprise a further genetic marker (or a combination of genetic markers) located in one of the coding sequence (e.g. exon) of the Dyf-7 gene or its ortholog is also included. In such embodiment, the genetic marker or combination of genetic markers located in one of the exons of the Dyf-7 gene or its ortholog can comprise, five, ten, fifteen, twenty, twenty-five or thirty additional genetic markers. In still another embodiment, besides the at least three polymorphic loci identified above and the at least one genetic marker located in the exon of the Dyf-7 gene or its ortholog, the genetic profile can comprise a further genetic marker (or a combination of genetic markers) located in one of the introns of the Dyf-7 gene or its ortholog. In such embodiment, the genetic marker or combination of genetic markers located in the introns of the Dyf-7 gene or its ortholog can comprise, twenty, thirty, forty, fifty or even a hundred additional genetic markers.

Once the profile has been determined, a correlation of the nematode's genetic profile with susceptible to macrocyclic lactone or resistance to macrocyclic lactone can then be made. This correlation is usually done by comparing the nematode's genetic profile obtained with a plurality of reference profiles. The reference profiles contain the genetic information of control nematodes for the marker(s) determined. A “susceptible” reference profile can contain for example, the nucleic acid identity of any one of the polymorphic loci identified in FIG. 9 which are more correlated to susceptibility than to resistance. An exemplary susceptibility profile (named susceptibility consensus) is provided in Table 3. A “resistance” reference profile can contain for example, the nucleic acid identity of the polymorphic loci identified in FIG. 9 which are more correlated to resistance than to susceptibility. An exemplary resistance profile (named resistance consensus) is provided in Table 3.

The marker(s) that is(are) being included in the genetic profile is not limited to a particular type of genetic polymorphism. For example, it can be single nucleotide polymorphisms (SNPs) identified in FIG. 9, an indel identified in FIG. 9, an allele derived from the polymorphic loci of FIG. 9 and/or a haplotype derived from the polymorphic loci of FIG. 9.

Profiles containing exclusively susceptibility-associated markers are indicators of high likelihood of susceptibility to macrocyclic lactones. On the other hand, profiles containing exclusively resistance-associated markers are indicators of high likelihood of resistance to macrocyclic lactones. However, some profiles can comprise both susceptibility-associated and resistance-associated markers. In these specific profiles, an analysis must be undertaken to weight the importance of each marker (or group of markers) with respect to susceptibility and resistance to determine if the profile is more likely associated with susceptibility or protection to macrocyclic lactones.

The methods described herein can be embodied in a system designed to perform the required steps. This system comprises at least two modules: a first module for performing the determination of the nematode's genetic profile and a second module for correlating the genetic profile to a susceptible/resistance phenotype towards macrocyclic lactones (e.g. a reference genetic profile). The first module comprises a detection module for determining the presence or absence of at least one marker identified in FIG. 9. The detection module can rely on the addition of a label to the sample and the quantification of the signal from the label for determining the presence or absence of the marker. The signal of the label is quantified by the detection module and is linked to the presence or absence of the marker. This label can directly or indirectly be linked to a quantifier specific for the marker. The information gathered by the detection module is then processed by the second module for determining the correlation. This second module can use a processor for comparing the genetic profile generated with the first module to a reference genetic profile (or a plurality of genetic profiles). The correlation module can then determine if the profile obtained from the determination module is more likely associated with susceptibility or resistance toward the macrocyclic lactones and as such, the nematode's susceptible/resistance phenotype towards macrocyclic lactones.

The markers are located in the Dyf-7 gene or its corresponding ortholog. The genetic profile can be determined at the genomic DNA level, at the messenger RNA level or at the protein level. Determination at the genomic DNA level is advantageous for determining the presence or absence of specific markers. When the determination is done at the genomic level, various assays can be used to determine the sequence of the marker. Such assays include, but are not limited to an allele-specific hybridization assay, an oligonucleotide ligation assay, an allele-specific elongation/ligation assay, an allele-specific amplification assay, a single-base extension assay, a molecular inversion probe assay, an invasive cleavage assay, a selective termination assay, restriction fragment length polymorphism (RFLP), a sequencing assay, single strand conformation polymorphism (SSCP), a mismatch-cleaving assay and denaturing gradient gel electrophoresis. It is worth indicating that it is not necessary to determine the sequence of the entire Dyf-7 gene (or its ortholog) or even the entire sequence of segment 4 of the Dyf-7 gene (or its ortholog) to determine the presence or absence of a particular marker. A fragment (as small as one nucleotide long and as long as the complete Dyf-7 gene minus one nucleotide) can also be sequenced to determine the presence or absence of the marker. If a fragment is sequenced, then it may be convenient to determine the position of the fragment that is being sequenced with respect to the Dyf-7 gene or the segment 4 of the Dyf-7 gene.

When the marker is associated with a genic region and its polymorphism can be detected in the transcript(s) of the gene comprising the marker, then the determination can be done at the messenger RNA level. At this level, it is first assessed whether the amount, concentration and/or nucleic acid sequence of a transcript in a nematode is different from those of a control. In order to do so, the skilled artisan can choose from many assays such as, for example, PCR, RT-PCR, microarray analysis and a sequencing assay.

When the marker is associated with a genic region and its polymorphism can be detected in a polypeptide encoded by a particular gene comprising the marker, then the determination of the profile can be done at the polypeptide level. Some markers will cause a differential splicing of transcript(s) of the polypeptide and as such will likely cause mutation(s) in the expressed polypeptide (truncation, localization, glycosylation pattern for example). When the determination is done at the polypeptide level and the marker induces a modification in the presentation of epitopes of the polypeptide, it may be advantageous to use an antibody or fragment thereof specific for the polypeptide. The determination at the polypeptide level can be done with various assays, such as, for example, ELISA, FACS analysis, Western blot, immunological staining assay, mass spectrometry, protein degradation and/or protein sequencing.

Since the methods described herein are based on the detection of alterations in the genomic DNA of the nematodes (and not transcripts or proteins), the methods are not limited to the characterization of adult nematodes. The methods can successfully be applied to nematodes at the larval stage and even to nematode eggs.

The methods presented herein can also be used to monitor macrocyclic lactone resistance in a population of nematodes. In order to do so, the method is practiced at a first point in time to determine, if any, the presence of macrocyclic lactone resistance in a population of nematodes. Then, the method is performed at least a second time, later in time, in order to determine if the phenotype of the population has changed.

The methods presented herein can also be used to determine if a subject intended to be treated with macrocyclic lactone or already being treated with macrocyclic lactone can benefit from a first or further macrocyclic lactone treatment. In order to do so, a biological sample from the subject suspected or known to be afflicted with a nematode infection is obtained either prior to macrocyclic lactone administration or after the administration of at least one dose of macrocyclic lactone. The genomic DNA of the nematodes contained in the biological sample is isolated and analyzed to characterize at least one of the polymorphic loci identified in FIG. 9. The presence of at least one polymorphism associated with susceptibility to macrocyclic lactone indicates that the subject will benefit from a macrocyclic lactone treatment (because the infecting nematodes are considered susceptible to macrocyclic lactone). In an embodiment, the method can also encompass administering macrocyclic lactone in such subjects. On the other hand, the presence of at least one polymorphism associated with resistance to macrocyclic lactone indicates that the subject will not benefit from a macrocyclic lactone treatment (because the infecting nematodes are considered resistant to macrocyclic lactone). In an embodiment, the method can also encompassing avoiding or discontinuing macrocyclic lactone treatment in such subject.

The methods presented herein can also be used to monitor the predisposition to macrocyclic lactone resistance in the treated subject. For example, the method can be practiced after the intake of a first dose of macrocyclic lactone by the treated subject to determine if the infecting nematodes are resistant or susceptible of developing a resistance against the macrocyclic lactone. In another example, the method can be practiced after the intake of a plurality of doses of macrocyclic lactone (and in some embodiment, during the entire period a macrocyclic lactone is administered to the infected subject) by the treated subject to determine if the infecting nematodes are resistant or susceptible of developing a resistance against the macrocyclic lactone.

The nucleic acid identity of at least one polymorphic locus can be determined by using a primer (or a plurality of primers). As used herein, a primer is an oligonucleotide used to initiate DNA replication. Typically, a primer is a short oligonucleotide that may be about 10, about 15, about 20, about 25, about 30, about 35, about 40, about 45, about 50, about 55, about 60, about 65, about 70, about 75, about 80, about 85, about 90, about 95, about 100 or more nucleotides. The primer-driven amplification is observed in PCR, allele-specific RT-PCR and qRT-PCR methods.

In an embodiment, a probe can be used for characterizing the at least one polymorphic locus identified in FIG. 9. The probe can be designed to specifically bind to a polymorphic locus or even to a specific polymorphism at this locus. The probe and the genomic DNA of the nematode that is being screened are placed into conditions favoring specific interactions (binding) between the probe and the genomic DNA. In the methods described herein, the formation (or lack of formation) of a complex between the probe and the genomic DNA of the nematode is thus indicative of the phenotype of the nematode. The probe may be, for example, an oligonucleotide, an aptamer or an antibody.

An “oligonucleotide” may comprise any size, shape and composition that is suitable for use in the context of the present disclosure. Preferably, an oligonucleotide of the present disclosure may comprise DNA, RNA, synthetic nucleotides, non-natural nucleotides, altered nucleotides, or combinations of one or more thereof. The term “oligonucleotide” refers to naturally-occurring species or synthetic species formed from naturally-occurring subunits or their close homologs. The term may also refer to moieties that function similarly to oligonucleotides, but have non-naturally-occurring portions. Thus, oligonucleotides may have altered sugar moieties or inter-sugar linkages. Exemplary among these are phosphorothioate and other sulfur containing species which are known in the art. In preferred embodiments, at least one of the phosphodiester bonds of the oligonucleotide has been substituted with a structure that functions to enhance the ability of the compositions to penetrate into the region of cells where the RNA whose activity is to be modulated is located. It is preferred that such substitutions comprise phosphorothioate bonds, methyl phosphonate bonds, or short chain alkyl or cycloalkyl structures. In accordance with other preferred embodiments, the phosphodiester bonds are substituted with structures which are, at once, substantially non-ionic and non-chiral, or with structures which are chiral and enantiomerically specific. Persons of ordinary skill in the art will be able to select other linkages for use in the practice of the present disclosure. Oligonucleotides may also include species that include at least some modified base forms. Thus, purines and pyrimidines other than those normally found in nature may be so employed. Similarly, modifications on the furanosyl portions of the nucleotide subunits may also be affected, as long as the essential tenets of the present disclosure are adhered to. Examples of such modifications are 2′-O-alkyl- and 2′-halogen-substituted nucleotides. Some non-limiting examples of modifications at the 2′ position of sugar moieties which are useful in the present disclosure include OH, SH, SCH₃, F, OCH₃, OCN, O(CH₂), NH₂ and O(CH₂)nCH₃, where n is from 1 to about 10. Such oligonucleotides are functionally interchangeable with natural oligonucleotides or synthesized oligonucleotides, which have one or more differences from the natural structure. All such analogs are comprehended by the present disclosure so long as they function effectively to specifically hybridize with the Dyf-7 gene (or gene ortholog) to detect the presence or absence of the genetic polymorphism. An oligonucleotide may be of any length that is suitable for use in methods of the present disclosure. In embodiments of the present disclosure, an oligonucleotide may comprise a sequence of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 250, or more nucleotides.

As used herein, an “aptamer” may be a nucleic acid or a peptide molecule that binds specifically to the Dyf-7 gene (or its gene ortholog) and allows for the detection of the presence or absence of the polymorphic locus/loci. For example, in solution, a chain of nucleotides may form intramolecular interactions that fold the aptamer into a complex three-dimensional shape. The shape of that aptamer allows it to bind tightly against the surface of its target molecule. Because of the diversity of molecular shapes that exists for nucleotide and amino acid sequences, aptamers may be obtained for a wide array of molecular targets, including, but not limited to, nucleic acid molecules, enzymes, membrane proteins, viral proteins, cytokines, growth factors, and immunoglobulins.

An aptamer may be a nucleic acid molecule. Said aptamer may comprise DNA, RNA, synthetic nucleotides, non-natural nucleotides, altered nucleotides, or combinations of one or more thereof. The nucleic acid aptamer may be single-stranded or double-stranded. A nucleic acid aptamer may comprise a sequence of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 250, at least 300, at least 350, at least 400, at least 500, or more nucleotides. A preferred nucleic acid aptamer may be a single stranded nucleic acid molecule and comprise a sequence of less than about 100 nucleotides. Alternatively, an aptamer may be a peptide molecule. A peptide aptamer may comprise a sequence of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200 or more amino acid residues. A preferred peptide aptamer may comprise a sequence of between about 15 to about 75 amino acid residues.

A probe of the present disclosure may be prepared according to standard techniques known to a skilled person. For example, a probe may be produced synthetically, recombinant or may be isolated from a natural source. In one embodiment, the source may be a biological source, for example, from a microorganism (e.g. a bacteria, a yeast or a virus), an animal (e.g. a mouse, a rat, a rabbit, a goat, or a human), or a plant.

In the context of the present disclosure, a probe may mean one probe or more than one probe. In one embodiment, a single probe may be used to characterize the polymorphisms. A skilled person would appreciate that one or more probes may be useful in the context of the present disclosure and may depend on the genotyping approach taken.

Probe design and production are known in the art. Generally, a probe may be produced recombinant, synthetically, or isolated from a natural source, e.g. from a cell, an animal, a yeast or a plant. However, a skilled person would appreciate that probe production may depend on the type of probe at issue.

### Tools and Commercial Packages for Detecting Macrocyclic Lactone Resistance

The methods described herein may be performed, for example, by utilizing pre-packaged diagnostic kits comprising at least one probe specific for a marker or for amplifying a fragment containing the marker, an antibody or fragment thereof specific for a polypeptide containing a marker.

In order to conduct the methods presented herewith, commercial packages are also provided. The commercial packages comprises means for characterizing the polymorphisms identified in FIG. 9 (and optionally listed in Table 3 or presented in FIG. 4) in a nematode. The commercial package can also comprise instructions for characterizing the phenotype of the nematode based on the nucleic acid identity at the polymorphic loci. Such instructions may comprise the information conveyed in the “characterizing” step of the methods described above.

The present invention will be more readily understood by referring to the following examples which are given to illustrate the invention rather than to limit its scope.

### Example

### Caenorhabditis elegans Culture Conditions

The wild-type Bristol N2 C. elegans strain was cultured in nematode growth medium (NGM) with OP50 Escherichia coli under standard conditions (Brenner, 1974). The IVR6 and IVR10 resistant strains to ivermectin were derived from Bristol N2, by successive increasing concentrations of ivermectin (James and Davey, 2009). The IVR6 and IVR10 ivermectin resistance strains, were cultured on NGM plates containing 6 ng/mL and 10 ng/mL of ivermectin respectively.

C. elegans Genomic DNA Preparation.

Three NGM plates from IVR6 and 5 plates from IVR10 strains with high numbers of nematodes were collected. The worms were soaked with M9 buffer and transferred into a 15 mL tube, followed by centrifugation at 1,500 g×3 min. The supernatant was removed and the nematode pellet was washed 5 times with M9 buffer following the same conditions. Then DNAse treatment was used to eliminate DNA contamination from OP50 E. coli or fungus present in the C. elegans cultures. Various aliquots from each strain containing a pellet of 100 μl of nematodes were treated with 6 U of rDNAse-I (AB Applied Biosystems), mixed well and incubated at 37° C. for 30 min. Then 10 μl of DNAse inactivation reagent were added, mixed well and incubated for 2 min at room temperature. The genomic DNA (gDNA) was extracted using the DNeasy™ Blood & Tissue kit (Qiagen) and visualized on 1% agarose gel contained 0.5 μg under UV illumination.

C. elegans Whole Genome Sequencing.

To sequence the whole genome of IVR6 and IVR10 strain nematodes, a high concentration and high quality of genomic DNA (gDNA) was required. From IVR6 (11 μg of gDNA) and IVR10 (6 μg of gDNA) were sequenced at McGill University/Genome Quebec Innovation Center by Illumina Genome Analyzer. Illumina single read libraries and 76 base sequencing lanes (one per sample) were generated, producing 1 Gb of base pair output per sample. The whole genome sequences from IVR6 and IVR10 were aligned with the last version (2008) of the whole genome sequence of C. elegans-Bristol N2 strain available at UCSC genome browser. The aligner software used was BWA (Li and Durbin 2009).

SNPs/Indels by Chromosome.

The data files were in “pileup format” that describes the base information at each chromosomal position. This format facilitates SNP/indel calling and alignment. The default output was generated by SAMtools (Li et al., 2009). SNP/indel data from IVR6 and IVR10 were generated together with SNP/indel data from the reference strain Bristol N2, making it possible to compare the SNP/indel variation from susceptible and resistant strains across the whole genome of the nematode. Due to the huge amount of SNP variation generated in the first data set, some filters were implemented to focus the search around the most significant and the best quality SNPs (coverage >20 and ≦50).

CT-Deletion in C. elegans Dyf7 Gene.

PCR was performed to confirm a deletion in the Dyf-7 gene. Primers Dyf7-CT-sense-5′-CCGTCGTAAGCAGAATTGAATC-3′ (SEQ ID NO: 1) and Dyf7-CT-antisense-5′-CATCATGTCAAAGGAGTCCTTC (SEQ ID NO: 2) were used to amplify a region in the Dyf7 gene in the wild type Bristol N2, IVR6 and IVR10 strains. Initial denaturation was performed at 94° C. for 3 min, followed by 37 cycles of 94° C. for 45 sec, 60° C. for 45 sec, 68° C. for 1 min, and the final extension was at 68° C. for 5 min. The PCR products were fractioned by electrophoresis on a 1% agarose gel, stained with ethidium bromide for ultraviolet visualization.

Rescue of Dyf7 Gene in IVR10.

Genomic DNA extracted from C. elegans C43C3 cosmid (provided by the Sanger Institute, UK) was used to amplify the Dyf7 gene using the primers Dyf7-CeB-sense-5′-GTCGAAACAGTAAATGAAAGAC-3′ (SEQ ID NO: 3) and Dyf7-CeB-antisense-5′-CTCATTTCTGGTCAAACG-3′ (SEQ ID NO: 4). A mixture of Dyf7 genomic PCR fragment of ˜5 Kb (including 2,000 Kb upstream and 500 bases downstream) at 10 ng/μL and ttx-3:GFP at 25 ng/μL was injected into IVR10 strain and two stable transgenic lines, with extrachromosomal arrays, were recovered. As a control, only ttx-3:GFP at 25 ng/μL was injected into IVR10 and two stable lines recovered. The worms were separated into transgenic and non-transgenic based on GFP expression (green channel) and then each population scored for resistance phenotype.

Haemonchus contortus Reference Strains/Isolates and DNA Extraction.

H. contortus macrocyclic lactone susceptible and resistant isolates (Mottier & Prichard, 2008) were used to identify single nucleotide polymorphic (SNP) sites in the Dyf7 gene. Additional reference isolates such us the drug-susceptible H. contortus CRA (Hc-CRA) from Republic of South Africa (RSA), and the multidrug-resistant isolates H. contortus Howick (RSA), Haecon 51 (Australia) and Roggliswill (Switzerland) were also included in this example. Genomic DNA was obtained from single adults H. contortus worms using the DNeasy™ Blood & Tissue kit, (Qiagen, Mississauga, ON, Canada), according to the manufacturer's protocol. Genomic DNA from H. contortus L3 larvae were extracted using QIAamp® DNA micro kit, followed for the whole genome DNA amplification using Repli-g® screening kit, according to the manufacturer's protocol.

Field H. contortus isolates with different geographical origins and with known drug efficacy phenotypic description were included in this example. The phenotypic description was according to the IVM treatment efficacy on a population of the isolate in the field obtained by fecal egg count reduction test (FECRT).

H. contortus Dyf7 Gene Amplification.

Since the whole H. contortus genome had not been published, H. contortus sequences were used, available from the Sanger Institute, and were annotated by Artemis software (Rutherford et al., 2000) using the homologous C. elegans Dyf7 gene as a reference to predict the open reading frame of the Dyf7 gene in H. contortus. In C. elegans, Dyf 7 gene is 2,497 nucleotides and is localized on chromosome X at positions 9698614-9701110, accession number NM_{—}077229.4 (UCSC Genome Browser, C. elegans 2008 assembly). Based on this information, optimal primers were designed using Geneious software, Primer 3 Program (Rozen and Skaletsky, 2000) to amplify the Dyf7 gene in H. contortus (Table 2).

TABLE 2
Oligonucleotides used to amplify the
whole Dyf-7 gene in Haemonchus contortus

| | | | Product |
|---|---|---|---|
| | | | size |
| Name (SEQ ID NO) | Oligonucleotide sequences (5′-3′) | Length | (bp) |
| Hc-dyf7-1-for (5) | TTGGAGTCAAAATCGACTGCGACGGCATT | 29 | 1130 |
| Hc-dyf7-1-rev (6) | TCACCAAAGCCGTCGGTATGTGCTTGTCG | 29 | |
| Hc-dyf7-2-for (7) | TTGGAGTCAAAATCGACTGCGACGGCATT | 29 | 1786 |
| Hc-dyf7-2-rev (8) | TGTGAGGCTTCCCGACGAGCATGACACCT | 29 | |
| Hc-dyf7-3-for (9) | TGCATTACTGTAGCCCAGGCTCT | 23 | 426 |
| Hc-dyf7-3-rev (10) | ACCGGTTGCGATTTTGGGTCGT | 22 | |
| Hc-dyf7-4-for (11) | TCTTTCCAGTGGACGAGGTGTCA | 23 | 690 |
| Hc-dyf7-4-rev (12) | AGAGGTCGTCCATCAGTGCTTCT | 23 | |
| Hc-dyf7-5-for (13) | GCGCTCGAAGACAATGTGGATACT | 24 | 638 |
| Hc-dyf7-5-rev (14) | ACACTTGTACCGTGCACTGAGCG | 23 | |
| Hc-dyf7-6-for (15) | TGCTATCACCCGATGCCCGA | 20 | 916 |
| Hc-dyf7-6-rev (16) | CCTTCTTCTGTTTCCATGTCGTCCA | 25 | |
| Hc-dyf7-7-for (17) | AGCAGTGAATCCGCCTTTTAGCA | 23 | 627 |
| Hc-dyf7-7-rev (18) | AGCATTAGCGGAGCTGGAATCCG | 23 | |
| Hc-dyf7-8-for (19) | TGTGTTGGGACCCGATTCTTAGCTAGT | 27 | 776 |
| Hc-dyf7-8-rev (20) | CCGAAGGTGACAGACAATGTCAAATGT | 27 | |

Polymerase chain reaction (PCR) was used to amplify each Dyf7 (Dyf7 1-8) segment. High Fidelity Platinum® Taq DNA polymerase (Invitrogen) was used in the PCR to increase the amplification accuracy. Initial denaturation was performed at 94° C. for 3 min, followed by 37 cycles of 94° C. for 45 sec, 60° C. for 45 sec, 68° C. for 1 min, and the final extension was at 68° C. for 5 min. The PCR products were fractioned by electrophoresis on a 1 agarose gel, stained with ethidium bromide for ultraviolet visualization. The amplification products were sequenced at McGill University/Genome Quebec Innovation Center. The sequence chromatograms were analyzed using the 4.9 version of the Sequencher software (Gene Codes Corporation, Ann Arbor, Mich. 48108, USA). Primary peaks and secondary peaks higher than 90% of the major nucleotide peak on the chromatogram were retained.

Quantitative Reverse Transcription-Polymerase Chain Reaction (qRT-PCR).

Total RNA was extracted from pools of L3 larvae from PF23 and MOF23 strains. Three different culture flasks for each strain were used in this example. The L3 larvae pools were homogenized in liquid nitrogen for 2 min, after that 1 mL of TRIzol® reagent (Invitrogen, USA) was added to the mortar, the homogenized solution was transferred to RNAase-free eppendorf tubes. The total RNA was extracted using the one-step method for TRIzol® reagent (Invitrogen, USA) according with the manufacture's protocol. RNA purity was determined spectrophotometrically and the RNA quality was assessed by electrophoresis in 1% agarose gel. One microgram of total RNA was treated with DNAse (gDNA Wipeout™ buffer, QuantiTect Reverse Transcription Kit, Qiagen, USA) and reverse transcribed to cDNA by using oligo dT from the same kit (Qiagen).

Quantitative RT-PCR was used to study the expression of Dyf7 gene in MOF23 in comparison with PF23 strain of H. contortus by the standard curve relative quantification method, that allows evaluation of the PCR efficiency. Quantitative RT-PCR was performed using gene-specific primers, that span exons 4 and 5, Dyf7-ex4-5-sense 5′-AGCCGAAACCGAAAGTAGAG-3′ (SEQ ID NO: 21) and Dyf7-ex4-5-antisense-5′-CGCTGAACATGCACTCTTTTG-3′ (SEQ ID NO: 22) and ‘quantitech SYBR-green master mix’ (Qiagen). The endogenous reference, 18s ribosomal-RNA (18srRNA) gene was amplified as described (Rao et al., 2009). All the qRT-PCR reactions were performed in a Rotor-Gene Q™ (Corbett Life Science, Qiagen) thermocycler. The amplification conditions included an initial denaturation step at 95° C. for 15 min, followed by 45 cycles of denaturation at 95° C. for 15 sec, annealing at 58° C. for 30 sec and extension at 72° C. for 30 sec. Melting curves were performed at a temperature range from 72-95° C. The amplicon specificity was assessed from the melting curve, showing a single peak, and by electrophoresis in a 2.5% agarose gel. Single qRT-PCR reactions were prepared at a final volume of 10 μL containing 5 μL of SYBR-green master mix, 2 μM of sense and antisense primers and 80-100 ng/μL of cDNA. Standard curves were produced to evaluate the PCR efficiency in the endogenous reference gene 18srRNA and in the gene of interest, Dyf7. A control-RT reactions containing 1 μg RNA, but no reverse transcriptase enzyme, was performed to survey possible gDNA contamination. PF23 cDNA (2,000 ng/μL) was 10-fold diluted to produce several dilutions standards from 2,000-0.02 ng. For each dilution point, four replicates were performed for each gene. Standard curves were generated by plotting the ΔCt against the log of cDNA dilutions, assessing the efficiency from the slope, and the r2 and r absolute values. Data was normalized to the endogenous reference 18srRNA gene using the ΔΔCt method (Livak and Schmittgen, 2001). Fold changes in gene expression were calculated using the comparative Ct method using the equation 2̂(−ΔΔCt) (Livak and Schmittgen, 2001). Experiments were repeated 3 times in four replicates for each sample.

H. contortus Dyf7-Intron Between Exons 7 and 8.

The genetic variation between susceptible (PF23) and resistant (MOF23 and IVF23) strains was investigated in the Dyf7-intron between exons 7 and 8, in 74 individual worms (PF23=23, MOF23=24, IVF23=27). The PCR amplification was performed using the primers Dyf7-sense-5′-GGTTTGGCAATCAAAGCACCGTCT-3′ (SEQ ID NO: 23) and Dyf7-antisense-5′-AAAGGCGGATTCACTGCTCTCT-3′ (SEQ ID NO: 24) following the same PCR conditions, and amplicon identification, described for the Dyf7 gene. Based on the sequencing data, single nucleotide polymorphisms were selected to evaluate the genetic variation between the H. contortus strains.

Statistical Analysis of the Allele Frequencies.

Variation in allele frequencies among strains was determined by analysis of molecular variance (AMOVA) using the computer program Arlequin version 2.000 (Schneider, Roessli and Excoffier, 2000). This program also estimated pairwise FST values and Slatkin's linearized FST[FST/1-FST] among populations and estimated the significance of the variance components associated with each level of genetic structure by nonparametric permutation test with 100,000 pseudoreplicates (Excoffier, Smouse and Quattro, 1992). Pairwise sequence alignments were used to construct a neighbor-joining NJ dendogram among populations using ClustalW 2.1 program (Larkin et al., 2007). Fisher's exact test and Student's t-test were estimated by using GraphPad Prism software (v 5.0 Window, GraphPad Software, Inc). Significance was based on a probability threshold of P<0.05.

Unexpected CT-Deletion in C. elegans Dyf7 Gene.

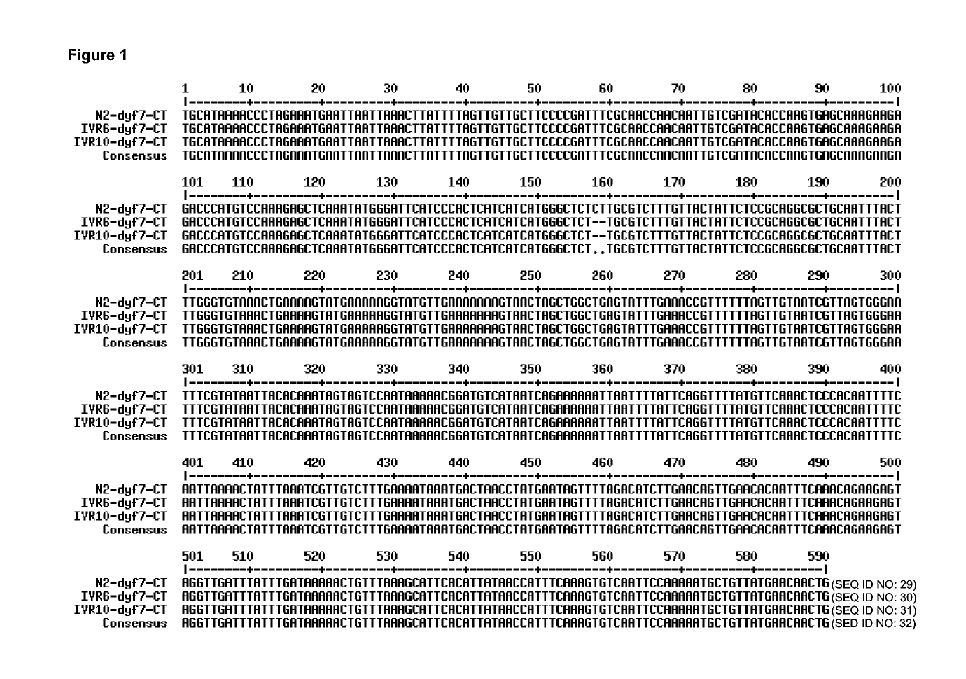

A PCR product of 690 bp was amplified in the wild type (Bristol N2), and in the resistant strains (IVR6 and IVR10). Sequencing results showed that a CT-deletion was only present in the two resistant strains IVR6 and IVR10 (FIG. 1). This would cause a frame shift and a nonsense translation product likely being responsible for the IVR6 and IVR10 resistance to ivermectin.

Rescue of Dyf7 Gene in IVR10.

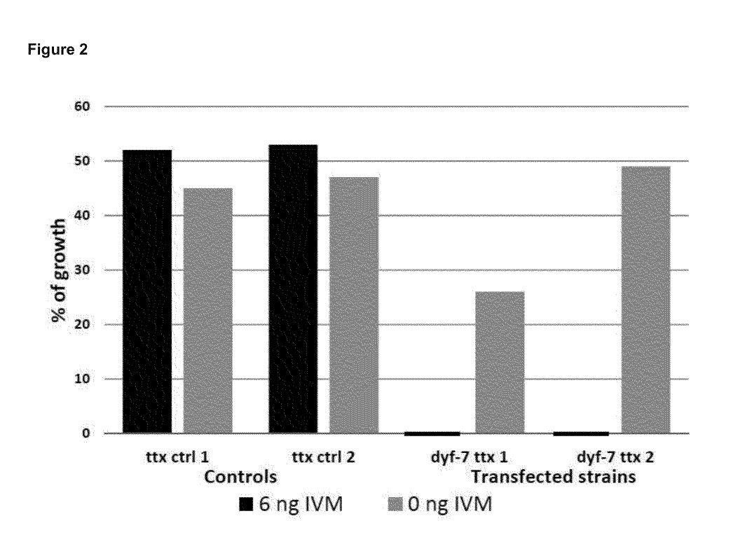

The CT-deletion suggests that this polymorphism is responsible for ivermectin resistance in C. elegans IVR6 and IVR10 strains. To determine if the Dyf7 rescuing fragment rescues the ivermectin-sensitivity phenotype of IVR10, each strain was plated, as eggs, onto NGM plates containing 6 ng/mL ivermectin and DMSO control plates. At two days, the L4 and adult worms were scored for presence of the transgene. The effect of Dyf7 rescued (strains Dyf7 ttx 1 and Dyf7 ttx 2) on ivermectin sensitivity is shown in FIG. 2. There were no transgenic worms that escaped L1 arrest in the Dyf7 ttx 1 and Dyf7 ttx 2 strains grown on 6 ng/mL ivermectin indicating that the wildtype Dyf7 transgene rescued the ivermectin sensitivity when inserted into the resistant strains.

Discovery of Single Polymorphic Nucleotide in the H. contortus Dyf7 Gene Homolog.

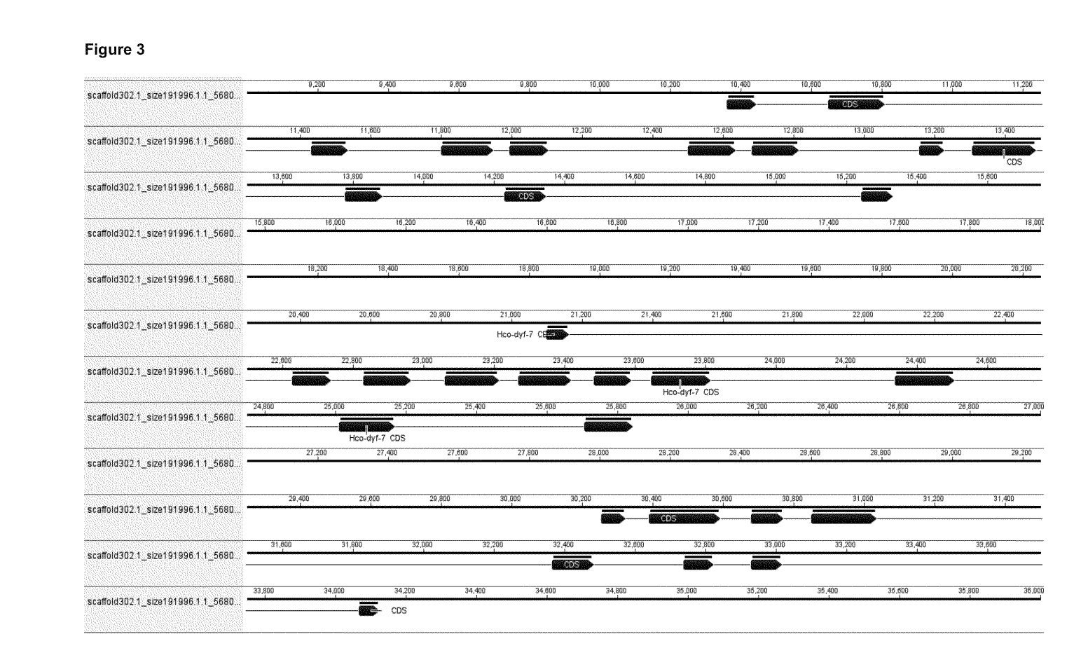

Table 1 lists the oligonucleotides used in this study to amplify Dyf7 gene in H. contortus and the predicted length of the PCR products. Dyf7 oligonucleotides were designed to amplify eight segments with overlap among them to allow the assembling of the whole segment sequences that contain a total of 10 exons and 9 introns (FIG. 3). The PF23-24 (susceptible isolate) and MOF23-9 (ML resistant isolate selected with moxidectin) were used as reference strains to amplify the 8 Dyf7 segments. Thirty one SNPs located in the coding region in Dyf7 gene clearly differentiated MOF23-9 from PF23-24. Once the location of a SNP was determined, it was assigned the name of the gene followed by a number indicating its distance in nucleotides from the adenine in the ATG start site. All the identified SNPs encoded synonymous substitutions in the samples analyzed in this study, nevertheless most of the SNPs clearly differentiated the reference isolates according to the known susceptible or resistant response to MLs.

Single Polymorphic Sites in Dyf7 Segment 4 from H. contortus.

From the 31 SNPs originally detected in the H. contortus Dyf7 gene, 17 SNPs were localized in Dyf7 segment 4 (Dyf7-4) (FIG. 4). The high number of polymorphisms was the main reason to choose this region for screening in more reference isolates and in samples from the field. Additionally, this segment of the gene contains exon-2, exon-3, exon-4 and partial exon-5. From the 17 SNPs, 15 SNP sites that were polymorphic in the reference and the field isolates are listed in Table 3 and were used as molecular markers to discriminate between susceptible and resistant phenotypes.

TABLE 3
Single nucleotide polymorphisms in Dyf7 gene, segment 4, in Haemonchus contortus
from different isolates. MOF23* reference isolate was derived from the unselected PF23
laboratory strain after 23 generations of moxidectin selection. Supplied by Fort Dodge Animal
Health. F15 from farm 15 in Brazil where failure to IVM has been observed. The polymorphic
loci are numbered from the cDNA of the PF23 strain, with the ‘A’ of the ATG start codon being
number 1.

| | | Isolate IVM Drug | No. individual | Locus | | | | | | | | | | | | | | |
|---|---|---|---|---|---|---|---|---|---|---|---|---|---|---|---|---|---|---|
| Strain | Country | Phenotype | worms | 141 | 153 | 192 | 234 | 246 | 267 | 288 | 303 | 309 | 312 | 396 | 402 | 438 | 447 | 462 |
| PF23-24 | USA | Susceptible | 1 | A | G | T | T | T | C | G | G | T | G | C | T | G | G | G |
| MOF23-9* | USA | Resistant | 1 | G | T | C | C | A | T | A | C | C | A | T | A | T | A | A |
| PF23 | USA | Susceptible | 18 | A | G | T | T | T | C | G | G | T | G | C | T | G | G | G |
| CRA | RSA | Susceptible | 1 | A | G | T | T | T | C | G | G | T | G | C | T | G | G | G |
| Sorin | France | Susceptible | 1 | A | G | T | T | T | C | G | G | T | G | C | T | G | G | G |
| Yallanbee | Australia | Susceptible | 1 | A | G | T | T | T | C | G | G | T | G | C | T | G | G | G |
| Wallangra | Australia | Susceptible | 1 | C | G | T | T | T | C | G | G | T | G | C | T | G | G | G |
| 85 | | | | | | | | | | | | | | | | | | |
| ISE | UK | Susceptible | 4 | A | G | T | T | T | C | G | G | T | G | C | T | G | G | G |
| MOF23 | USA | Moderate Resistant | 10 | A | G | T | T | T | C | G | G | T | G | C | T | G | G | G |
| | | | 2 | R | G | T | T | T | C | G | G | T | G | C | W | G | G | G |
| | | | 3 | R | G | B | Y | A | H | A | C | C | A | T | A | — | — | — |
| | | | 28 | G | T | C | C | A | T | A | C | C | A | T | A | T | A | A |
| F15 | Brazil | Resistant | 1 | A | G | T | T | T | C | G | G | T | G | C | T | G | G | G |
| | | | 2 | G | T | C | C | A | T | A | C | C | A | T | A | T | A | A |
| 1202 | Brazil | Resistant | 1 | G | T | C | C | T | C | G | C | C | G | T | A | T | A | A |
| Coruzu | Argentina | Resistant | 1 | G | T | C | C | A | T | A | C | C | A | T | A | T | A | A |
| SUL | Uruguay | Resistant | 1 | G | T | C | C | A | C | A | G | T | A | T | A | T | G | G |
| | | | 1 | G | T | C | C | A | C | A | G | T | A | T | A | T | G | G |
| Haecon | Australia | Resistant | 1 | G | T | C | C | A | T | A | C | C | A | T | A | T | G | G |
| Rog-gliswil | Switzer- | Resistant | 1 | G | T | C | C | T | C | G | C | C | G | T | A | T | A | A |
| | land | | | | | | | | | | | | | | | | | |
| Cedara | RSA | Resistant | 1 | G | G | T | C | A | C | G | C | C | G | C | T | T | G | G |
| | | | 2 | G | T | C | C | A | T | A | C | C | A | T | A | T | A | A |
| Howick | RSA | Resistant | 1 | G | T | C | C | A | T | A | C | C | A | T | A | T | A | A |
| Kokstad | RSA | Resistant | 2 | G | G | T | C | A | C | G | C | C | G | C | T | T | G | G |
| | | | 1 | G | T | C | C | A | T | G | C | T | A | T | A | T | G | G |
| | | | 1 | G | T | C | C | A | T | A | G | T | A | T | A | T | A | A |
| | | | 1 | G | T | C | C | A | T | A | C | C | A | T | A | T | A | A |

| | | | | | | | | | | | | | | | |
|---|---|---|---|---|---|---|---|---|---|---|---|---|---|---|---|
| Identity most associated with resistant | G | T | C | C | A | T | A | C | C | A | T | A | T | A | A |
| Identity most associated with susceptible | A | G | T | T | T | C | G | G | T | G | C | T | G | G | G |

A 690 bp fragment of Dyf7-4 was amplified and sequenced in 79 H. contortus isolates from different geographical origins with known drug efficacy in the field (Table 3). The PF23 (USA), CRA (RSA), Sorin (France), Wallangra 1985 (Australia), ISE (United Kingdom), with known susceptible response to MLs (Mottier & Prichard, 2008; Rufener et al., 2009) showed identical nucleotides at each of the 15 SNP positions. The MOF23 isolate, selected with moxidectin (and resistant to IVM), showed a different nucleotide at each of these SNP sites, in comparison with PF23-24 and the remaining susceptible isolates. A majority of worms from the H. contortus isolates MOF23 (USA), F15 and 1202 (Brazil), Coruzu (Argentina), SUL (Uruguay), Haecon (Australia), Roggliswil (Switzerland), Cedera, Howick, and Kokstad (South Africa), which had overall IVM resistant phenotypes, showed most of the 15 SNPs found in the MOF23-9 sequence. While isolates may show an overall resistant phenotype, it does not necessarily mean that every individual worm from that isolate will have the resistant genotype. The efficacy of the drug in the field, against each isolate, had been assessed by the FECRT, which is not a very sensitive method and may not give an accurate assessment of the proportion of the isolate that is anthelmintic resistant (Cabaret & Berragb, 2004). Due to the high agreement between the drug response phenotype and the change in polymorphism in many of these 15 SNPs, it is suggested that each of these 15 SNP markers, or a combination of two or more of these polymorphic sites, are potential candidates for ML drug resistant surveillance in the field. In particular, the SNPs at positions 141, 234 and 438, which were present in all of the worms that were genotyped from the resistant isolates that deviated significantly from the wild-type (susceptible genotype) are strong markers for ML resistance when assayed individually or as a genotype combination. As the sequences indicated in Table 3, FIG. 4 and FIG. 9 commence at different positions in the gene and are accordingly numbered differently, Table 4 relates the location for each SNP as described in Table 3, FIG. 4 and those identified in FIG. 9 which correspond to the SNPs identified in FIG. 4 or Table 3.

TABLE 4
Single nucleotide polymorphism identity shown in Table 3 and the
corresponding location in FIG. 4 and FIG. 9. ^{¶}SNP not included in
Table 3, but included in FIG. 4 and FIG. 9. ^{†}Additional SNP differences
identified between dyf7-PF23 and dyf7-MOF23, shown in FIG. 9, are
listed in the text.

| | Equivalent | Equivalent |
|---|---|---|
| SNP identity | SNP location | SNP location |
| in Table 3 | in FIG. 4 | in FIG. 9^{†} |
| 141 (A-G) | 407 | 1769 |
| 153 (G-T) | 419 | 1781 |
| 192 (T-C) | 561 | 1923 |
| 234 (T-C) | 588 | 1950 |
| 246 (T-C) | 603 | 1965 |
| 267 (C-T) | 615 | 1977 |
| 288 (G-A) | 636 | 1998 |
| 303 (G-C) | 764 | 2126 |
| 309 (T-C) | 779 | 2141 |
| 312 (G-A) | 785 | 2147 |
| 396 (C-T) | 788 | 2150 |
| 402 (T-A) | 834 | 2196 |
| 438 (G-T) | 872 | 2234 |
| 447 (G-A) | 878 | 2240 |
| 462 (G-A) | 979 | 2341 |
| N/A | 988 | 2350 |
| N/A | 1003 | 2365 |
| N/A | 1027 | 2389 |
| N/A | 1036 | 2398 |
| N/A | 1060 | 2422 |
| N/A | 1078 | 2440 |

When the frequency of the Dyf-7 gene containing the resistance associated SNPs in a population of H. contortus was compared with Hardy-Weinberg equilibrium, there appeared to be an excess of homozygosity, which could be explained if the Dyf-7 gene is sex-linked and thus hemizygous in XO males. Therefore, 55 adult H. contorus, whose sex could be easily ascertained, were individually genotyped for the presence or absence of the resistance SNPs. Of 29 adult MOF23 males that were genotyped, none was heterozygous, whereas 26 MOF23 female H. contortus were in Hardy-Weinberg equilibrium (8 ss, 11 rs, 7 rr, where s=a susceptible haplotype and r=a resistant haplotype). These results are consistent with the Dyf-7 gene being sex-linked in a XO male/XX female organism (p<1×10−6). Knowledge that the resistance-associated SNPs in H. contortus is sex-linked increases the predictive value of the diagnostic test for resistance and the value of undertaking diagnosis of the resistance status of a nematode population in the management of resistance. The resistance genotype frequency and thus the resistance phenotype will increase more rapidly in a population of nematode parasites with anthelmintic selection pressure if the resistance is sex-linked than if the resistance is autosomal.

Dyf7 Gene Expression in H. contortus.

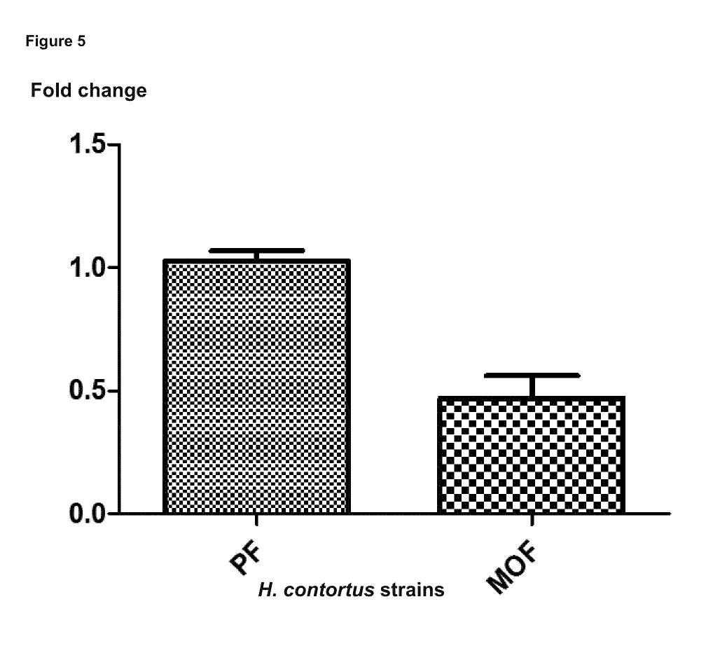

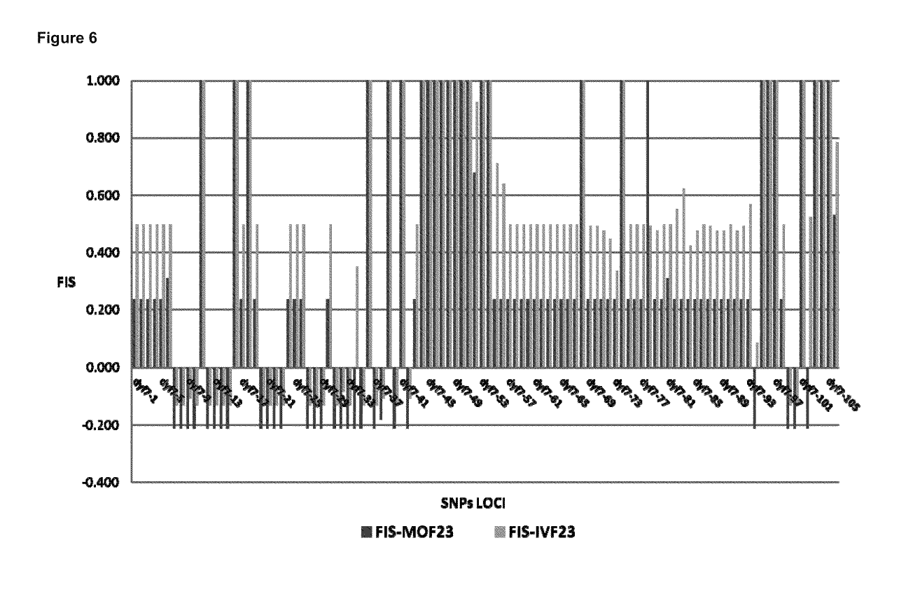

The expression level of the Dyf7 gene in PF23 (susceptible) and MOF23 (resistant) strains was assessed by qRT-PCR. Data was normalized to the endogenous reference 18srRNA gene. FIG. 5 shows the fold changes in the Dyf7 expression between the susceptible and resistant strains. The expression of Dyf7 gene was significantly reduced in the resistant MOF23 strain compared with the unselected (susceptible) PF23 parental strain (P=0.005). It is hypothesized that the decrease in level of Dyf7/8 mRNA will lead to a change and decrease in level of protein expression of Dyf7/8.

H. contortus Dyf7 Intron Spanning Exons 7 and 8.

A 643-bp intronic region of Dyf7 gene was amplified and surveyed for single nucleotide polymorphisms. From this region 106 SNP loci were selected to assess the genetic variation among PF23, MOF23 and IVF23 (ivermectin selected from the PF strain and IVM resistant) populations. Wright's FIS was estimated for each locus in each population by the method of Weir and Cockerham (Weir and Cockerham, 1984) to test for Hardy-Weinberg proportions among genotypes where FIS=1−(heterozygotes observed/heterozygotes expected). If an excess of heterozygotes are observed, then FIS<0 and if an excess of homozygotes are observed, then FIS>0. A frequency histogram of population specific FIS across each polymorphic site is shown in FIG. 6. The PF23 strain was homozygous for most of the 106 loci analyzed. For MOF23 and IVF23 these loci showed a consistent excess of homozygotes of 74% and 76%, respectively, for the alternative, resistance associated nucleotide, when compared with Hardy-Weinberg expectations. The excess of homozygotes (for the alternative nucleotide) in these loci in the ML selected strains is consistent with drug selection and provides further evidence that the Dyf7 gene in nematodes is under selection when ML resistance arises.

SNPs Allele Frequencies Among PF23, MOF23 and IVF23 Populations.

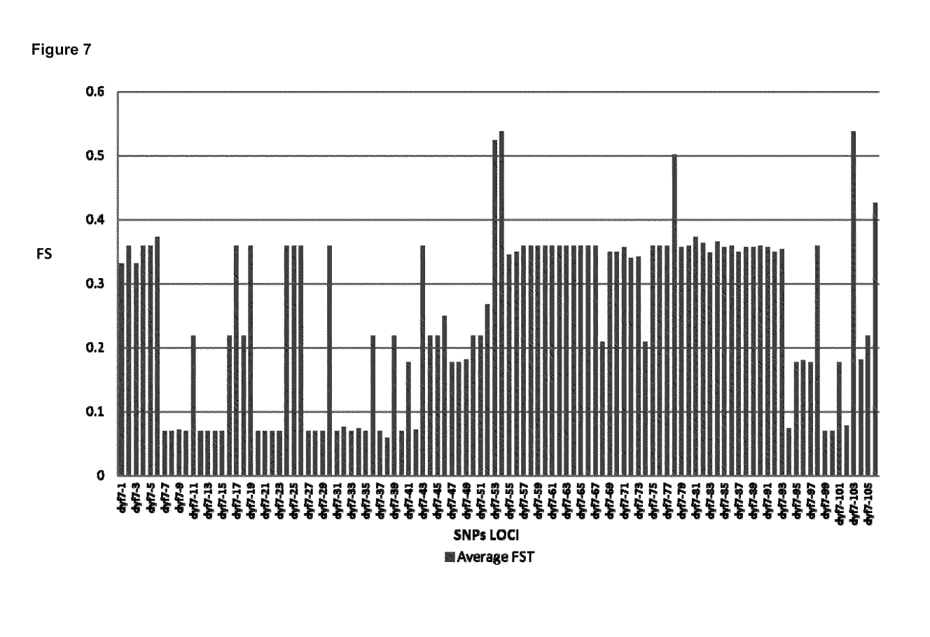

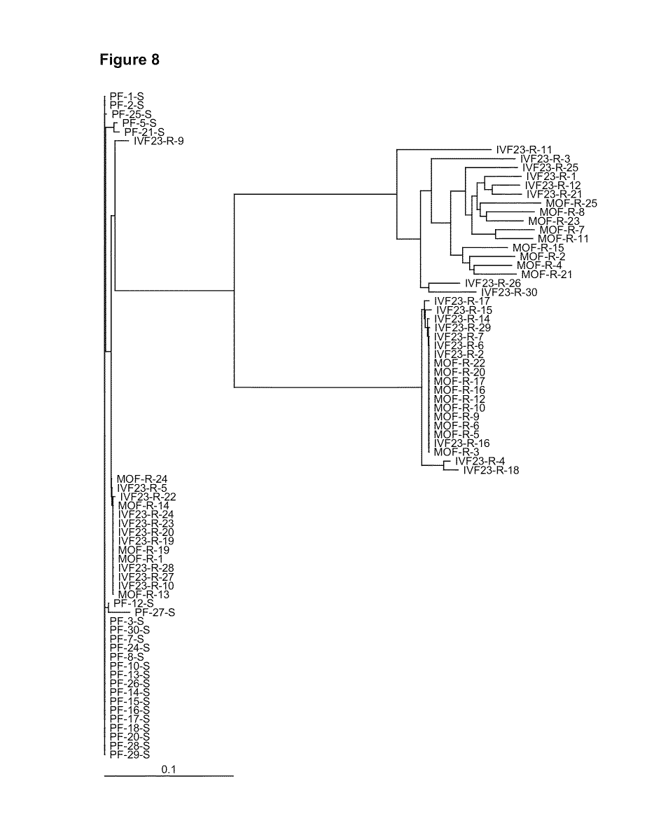

The Weir and Cockerham estimate of Wright's FST was calculated for each locus. The FST ranged between 0.062-0.528 at the 106 loci with an average of 0.249 (FIG. 7). Variation in the SNP allele frequencies were compared among and within of the three populations by AMOVA. The highest variation (38%) of the SNP allele frequencies was attributable to within individual worms in a population, followed by 33% of variation among individuals worms in a population, and 29% was caused by variation among the three populations. The difference among the variance components associated with each level of genetic structure was highly significant (P<0.000001). A cluster analysis of pairwise sequence alignments among PF23, MOF23 and IVF23 populations based upon 106 SNP loci is showed in FIG. 8. The variance of the SNP allele frequencies among the three populations shows a first cluster, which is less variable, located near to the root of the tree that contains all the PF23 population and some MOF23 and IVF23 individuals. A second cluster arising, with a higher variability, and is subdivided into two branches that contain indistinctively MOF23 and IVF23 populations. The first branch shows more variability among MOF23 and IVR23 isolates when we compare with the second branch.

Sequence Information of the Polymorphic Loci Identified.

FIG. 9 provides a sequence comparison of the genomic (gDNA) and cDNA contig of two H. contortus strains (susceptible=PF23 and resistant=MOF23). In the this figure, the various SNPs are identified with a Δ, whereas the indels are identified with →.

### REFERENCES

Brenner, S. 1974. The genetics of Caenorhabditis elegans. Genetics 77, 71-94.
Cabaret J, Berragb B. 2004. Faecal egg count reduction test for assessing anthelmintic efficacy: average versus individually based estimations. Veterinary Parasitology 121:105-113.
Excoffier, L., Smouse, P. E., Quattro, J. M. 1992. Analysis of molecular variance inferred from metric distances among DNA haplotypes: application to human mitochondrial DNA restriction data. Genetics. 131(2): 479-91.
James, C. E., Davey, M. W. 2009. Increased expression of ABC transport proteins is associated with ivermectin resistance in the model nematode Caenorhabditis elegans. Int. J. Parasitol. 39, 435: 213-220.
Larkin E K, Morris N J, Li Y, Nock N L, Stein C M. 2007. Comparison of affected sibling-pair linkage methods to identify gene x gene interaction in GAW15 simulated data. BMC Proc. 1 Suppl 1: S66.
Li, H. and Durbin, R. 2009. Fast and accurate short read alignment with Burrows—Wheeler transform. Bioinformatics. Vol. 25 no. 14: 754-1760.
Li, H., Handsaker, B., Wysoker, A., Fennell, T., Ruan, J., Homer, N., Marth, G., Abecasis, G., Durbin R. and 1000 Genome Project Data Processing Subgroup. 2009. The Sequence alignment/map (SAM) format and SAMtools. Bioinformatics, 25, 2078-9. Livak and Schmittgen, 2001.
Mottier, M. L., Prichard, R. K. 2008. Genetic analysis of a relationship between macrocyclic lactone and benzimidazole anthelmintic selection on Haemonchus contortus. Pharmacogenetics and Genomics 18: 129-140.
Rao, V. T., Siddiqui, S. Z., Prichard, R. K., Forrester, S. G. 2009. A dopamine-gated ion channel (HcGGR3*) from Haemonchus contortus is expressed in the cervical papillae and is associated with macrocyclic lactone resistance. Mol Biochem Parasitol 166 54-61.
Rozen, S. and Skaletsky, H. 2000. Primer3 on the WWW for general users and for biologist programmers. In: S. Krawetz, S. Misener, eds. Bioinformatics Methods and Protocols in the series Methods in Molecular Biology. Humana Press, Totowa, N.J. 365-386.
Rufener L, Maser P, Roditi I, Kaminsky R. 2009. Haemonchus contortus Acetylcholine Receptors of the DEG-3 Subfamily and Their Role in Sensitivity to Monepantel. PLoS Pathog 5(4): e1000380.
Rutherford. K., Parkhill, J., Crook, J., Horsnell, T., Rice, P., Rajandream, M. A. and Barrell, B. 2000. Artemis: sequence visualization and annotation. Bioinformatics (Oxford, England). 16.10: 944-5.
Schneider S, Roessli D and Excoffier L. 2000. Arlequin: a software for population genetics data analysis. User manual ver 2.000. Genetics and Biometry Lab, Dept. of Anthropology, University of Geneva, Geneva.
Weir B S, Cockerham C C. 1984. Estimating F-statistics for the analysis of population structure. Evolution Int J Org Evolution 38:13587-1370.

What is claimed is:

1. A method for assessing the susceptibility of a nematode to a macrocyclic lactone, said method comprising:
(a) providing a genomic DNA sample of the nematode comprising a Dyf-7 gene or a Dyf-7 gene ortholog;
(b) determining the nucleic acid identity of at least one polymorphic locus identified in FIG. 9 in the genomic DNA;
(c) associating the nucleic acid identity obtained in step (b) either to the nucleic acid identity of a corresponding at least one polymorphic locus of a resistant phenotype or with the nucleic acid identity of a corresponding at least one polymorphic locus of a susceptible phenotype; and
(d) characterizing the nematode as being (i) susceptible to the macrocyclic lactone when the nucleic acid identity of the at least one polymorphic locus is determined to be associated with the susceptible phenotype or (ii) resistant to the macrocyclic lactone when the nucleic acid identity of the at least one polymorphic locus is determined to be associated with the resistant phenotype.

2. The method of claim 1, wherein the at least on polymorphic locus is listed in Table 3.

3. The method of claim 1, wherein the macrocyclic lactone is ivermectin.

4. The method of claim 1, wherein the nematode is from a Trichostrongylidae family.

5. The method of claim 4, wherein the nematode is from a Haemonchus genus.

6. The method of claim 1, wherein the nematode is an adult nematode, a larva or an egg.

7. The method of claim 1, further comprising, prior to step (b), providing a nucleic acid synthetic copy of the genomic DNA comprising the Dyf-7 gene or the Dyf-7 gene ortholog and using the nucleic acid synthetic copy to determine the nucleic acid identity.

8. The method of claim 7, further comprising, prior to step (b), amplifying a portion of the Dyf-7 gene or the Dyf-7 gene ortholog corresponding to the portion of the Dyf-7 gene amplified with a pair of primers comprising a first primer having the following nucleic acid sequence TCTTTCCAGTGGACGAGGTGTCA (SEQ ID NO: 11) and a second primer having the following nucleic acid sequence AGAGGTCGTCCATCAGTGCTTCT (SEQ ID NO: 12) to provide the nucleic acid synthetic copy.

9. The method of claim 1, wherein the at least one polymorphic locus comprises Hco-Dyf7-141, Hco-Dyf7-234 and/or Hco-Dyf7-438.

10. The method of claim 1, further comprising, in step (b), determining the nucleic acid identity of at least three polymorphic loci.

11. The method of claim 10, wherein the at least three polymorphic loci comprise at least one of Hco-Dyf7-141, Hco-Dyf7-234 or Hco-Dyf7-438.

12. A commercial package for the detection of macrocyclic lactone resistance in a nematode, said commercial package comprising means for determining the nucleic acid identity of at least one polymorphic locus identified in FIG. 9 and instructions for characterizing the nematode based on the nucleic acid identity.

13. A method for assessing the susceptibility of a nematode to a macrocyclic lactone, said method comprising:
(a) providing a gene product from a Dyf-7 gene or a Dyf-7 gene ortholog from the nematode;
(b) determining the test level of the gene product;
(c) comparing the test level of the gene product to a control level of the gene product of a Dyf-7 gene or a Dyf-7 gene ortholog associated with susceptibility to the macrocyclic lactone; and
(d) characterizing the nematode as susceptible to the macrocyclic lactone when the test level is determined to be equal to or higher than the control level and as resistant to the macrocyclic lactone when the test level is determined to be lower than the control level.

14. The method of claim 13, wherein the macrocyclic lactone is ivermectin.

15. The method of claim 13, wherein the nematode is from a Trichostrongylidae family.

16. The method of claim 15, wherein the nematode is from a Haemonchus genus.

17. The method of claim 13, wherein the nematode is an adult nematode, a larva or an egg.

18. The method of claim 13, wherein the gene product is a transcript of the Dyf-7 gene or the Dyf-7 gene ortholog.

19. The method of claim 13, wherein the gene product is a protein encoded by the Dyf-7 gene or the Dyf-7 gene ortholog.
